SEQUENCE LISTING (13 sequences)

SEQ ID NO: 1 - protein, 5 aa, Artificial sequence
  VH CDR1
NYALN

SEQ ID NO: 2 - protein, 17 aa, Artificial sequence
  VH CDR2
WINTYSGVGTYAGEFKG

SEQ ID NO: 3 - protein, 11 aa, Artificial sequence
  VH CDR3
RGFYGRRFFDV

SEQ ID NO: 4 - protein, 120 aa, Artificial sequence
  Heavy chain sequence
QVQLVQSGSELKKPGASVKVSCKASGYTFTNYALNWVRQAPGQGLEWMGWINTYSGVGTYAGEFKGRFVFSLDTSVSTAYLQISSLKAEDTAVYYCARRGFYGRRFFDVWGKGTTVTVSS

SEQ ID NO: 5 - protein, 15 aa, Artificial seqeunce
  VL CDR1
KASQSVDYDVDSYMH

SEQ ID NO: 6 - protein, 7 aa, Artificial sequence
  VL CDR2
AASNLAS

SEQ ID NO: 7 - protein, 9 aa, Artificial sequence
  VL CDR3
QQTIEDPPT

SEQ ID NO: 8 - protein, 111 aa, Artificial sequence
  Light chain sequence
DIQLTQSPSSLSASVGDRVTITCKASQSVDYDVDSYMHWYQQKPGKAPKLLIYAASNLASGVPSRFSGSGSGTDFTFTISSLQPEDIATYYCQQTIEDPPTFGGGTKVEIK

SEQ ID NO: 9 - protein, 447 aa, Artificial sequence
  Full length heavy chain
QVQLVQSGSELKKPGASVKVSCKASGYTFTNYALNWVRQAPGQGLEWMGWINTYSGVGTYAGEFKGRFVFSLDTSVSTAYLQISSLKAEDTAVYYCARRGFYGRRFFDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 218 aa, Artificial sequence
  Full length light chain
DIQLTQSPSSLSASVGDRVTITCKASQSVDYDVDSYMHWYQQKPGKAPKLLIYAASNLASGVPSRFSGSGSGTDFTFTISSLQPEDIATYYCQQTIEDPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 107 aa, Homo sapiens [sequence omitted]

SEQ ID NO: 13 - protein, 480 aa, Homo sapiens
SPVPFGPEGRLEDKLHKPKATQTEVKPSVRFNLRTSKDPEHEGCYLSVGHSQPLEDCSFNMTAKTFFIIHGWTMSGIFENWLHKLVSALHTREKDANVVVVDWLPLAHQLYTDAVNNTRVVGHSIARMLDWLQEKDDFSLGNVHLIGYSLGAHVAGYAGNFVKGTVGRITGLDPAGPMFEGADIHKRLSPDDADFVDVLHTYTRSFGLSIGIQMPVGHIDIYPNGGDFQPGCGLNDVLGSIAYGTITEVVKCEHERAVHLFVDSLVNQDKPSFAFQCTDSNRFKKGICLSCRKNRCNSIGYNAKKMRNKRNSKMYLKTRAGMPFRVYHYQMKIHVFSYKNMGEIEPTFYVTLYGTNADSQTLPLEIVERIEQNATNTFLVYTEEDLGDLLKIQLTWEGASQSWYNLWKEFRSYLSQPRNPGRELNIRRIRVKSGETQRKLTFCTEDPENTSISPGRELWFRKCRDGWRMKNETSPTVELP

(19) United States
(12) Patent Application Publication
(10) Pub. No.: US 2023/0131324 A1
(43) Pub. Date: Apr. 27, 2023

(54) FORMULATIONS OF ANTI-ENDOTHELIAL LIPASE ANTIBODIES

(71) Applicant: MEDIMMUNE, LLC, Gaithersburg, MD (US)

(72) Inventors: Sajal Manubhai PATEL, Germantown, MD (US); Swapnil Khushal PANSARE, Monroe Township, NJ (US)

(21) Appl. No.: 17/801,670

(22) PCT Filed: Feb. 23, 2021
(86) PCT No.: PCT/US21/19213

§ 371 Date: Aug. 23, 2022

Related U.S. Application Data
(60) Provisional application No. 62/980,658, filed on Feb. 24, 2020.

(51) Int. Cl. A61K 39/395; C07K 16/40; A61K 47/18; A61K 47/22; A61K 47/26; A61K 9/00; A61K 9/19; A61P 9/00; A61P 9/10

(52) U.S. Cl. CPC A61K 39/39591; C07K 16/40; A61K 47/183; A61K 47/22; A61K 47/26; A61K 9/0019; A61K 9/19; A61P 9/00; A61P 9/10; C07K 2317/76; A61K 2039/545

(57) ABSTRACT

Provided are formulations of an antibody that specifically binds of endothelial lipase. The provided formulations may be in liquid form or reconstituted from a lyophilized form. The formulations have viscosities suitable for injection and prevent reversible self-association of the antibody, precipitation and cloudiness.

### FIELD OF THE INVENTION

The present technology relates generally to formulations of antibodies to anti-endothelial lipase. The formulations are stable and reduce undesirable drug product characteristics, such as reversible self-association, precipitation and cloudiness. The formulations also have viscosities suitable for injection either manually or with automated injection systems.

### BACKGROUND OF THE INVENTION

Endothelial lipase is a phospholipase of the triglyceride lipase family that catalyzes the conversion of high-density lipoprotein (HDL) to low-density lipoprotein (LDL). Jaye et al., Nature Genetics 21:424-428 (1999). HDL is informally referred to as “good cholesterol” and has been shown to transport fat molecules out of artery walls, reduce macrophage accumulation, and therefore help prevent or even regress atherosclerosis (the narrowing of arteries). Humanized monoclonal antibodies inhibiting the activity of endothelial lipase have been developed, examples of which are described in US Application Publication No. 2017/0260290.

Increased plasma endothelial lipase concentrations have been associated with a deteriorated lipoprotein-lipid profile along with elevated plasma triglyceride and apolipoprotein B concentrations, as well as with smaller LDL particle size. (Paradis et al., Can J Cardiol 22: 31B-34B (2006).) Elevated proinflammatory cytokine concentrations and an increased prevalence of the metabolic syndrome have also been observed among individuals with elevated plasma endothelial lipase concentrations. (Id.) Given these and other factors, endothelial lipase has been considered to play an important role in cardiovascular disease. (Id.)

Despite the effectiveness of current therapies for cardiovascular disease such as high potency statins, there remains a significant residual risk of major adverse cardiovascular events in patients with acute coronary syndrome. The majority of myocardial infarctions occur in patients with normal LDL levels, and despite treatment with high dose and highly potent statins, PCSK9 inhibitors, and/or ezetimibe following a myocardial infarction, the residual risk of a second cardiovascular event remains high. For example, the IMPROVE-IT study (ezetimibe+statin) had a 33% risk of a cardiovascular event with 7 years of follow-up. In addition, ODYSSEY (PCSK9 inhibitor+statin) has a 9.5% residual risk over 2.8 years.

Several attempts to pharmacologically raise HDL levels using different mechanisms of action were found to not significantly improve cardiovascular outcomes. In particular, four trials of cholesterol transfer protein (CETP) inhibitors have been completed. While CETP inhibitors raise HDL cholesterol, three of the four trials did not improve cardiovascular outcomes, and in the one trial that did reduce cardiovascular events, the effect was modest with only a 9% relative risk reduction. This approach has been criticized since the inhibition of CETP causes a block in LDL receptor-mediated reverse cholesterol transport.

Humans with partial and complete loss of function mutations in the gene encoding endothelial lipase exhibit elevated HDL cholesterol, increased cholesterol efflux capacity, and trends towards reduced cardiovascular risk. Thus, neutralization of EL represents a promising therapeutic mechanism.

Biological pharmaceutical products, often called biologics, are frequently administered via injection. Solutions of large molecular weight biologics, such as monoclonal antibodies, typically have high concentrations and high viscosities that can lead to issues with product suitability and injection delivery. Particular suitability concerns for antibodies include reversable self-association (the formation of oligomeric species as a result of noncovalent intermolecular interactions), precipitation of protein and the osmolality and cloudiness of the solution. Viscosity and suitability can affect manufacturing, stability, delivery, and safety of the biologic. Thus, there is a need in the art for suitable formulations of biologics, such as anti-endothelial lipase antibodies, having suitable concentrations of active agent and viscosities suitable for injection.

### BRIEF SUMMARY OF THE INVENTION

The present disclosure is directed to a pharmaceutical formulation comprising: (i) about 40 mg/mL to about 160 mg/mL of an antibody that specifically binds to human endothelial lipase, and (ii) about 50 mM to about 260 mM arginine salt. In some embodiments, the pharmaceutical formulation comprises about 125 mg/mL of the antibody that specifically binds to endothelial lipase. In some embodiments, the pharmaceutical formulation comprises about 100 mg/mL of the antibody that specifically binds to endothelial lipase.

In some embodiments of the pharmaceutical formulation, the arginine salt comprises arginine hydrochloride, arginine acetate, arginine glutamate, or arginine sulfate. In some embodiments, the pharmaceutical formulation comprises arginine hydrochloride. In some embodiments, the pharmaceutical formulation comprises about 220 mM arginine hydrochloride. In some embodiments, the pharmaceutical formulation comprises about 160 mM arginine hydrochloride. In some embodiments, the pharmaceutical formulation comprises about 80 mM arginine hydrochloride.

In some embodiments of the pharmaceutical formulation, the antibody that specifically binds to endothelial lipase has a first heavy chain complementarity determining region (CDR1) with an amino acid sequence at least 80% identical to SEQ ID NO: 1. In some embodiments of the pharmaceutical formulation, the antibody that specifically binds to endothelial lipase has a heavy chain CDR1 comprising SEQ ID NO: 1. In some embodiments of the pharmaceutical formulation, the antibody that specifically binds to endothelial lipase has a second heavy chain complementarity determining region (CDR2) with an amino acid sequence at least 90% identical to SEQ ID NO: 2. In some embodiments of the pharmaceutical formulation, the antibody that specifically binds to endothelial lipase has a heavy chain CDR2 comprising SEQ ID NO:2. In some embodiments of the pharmaceutical formulation, the antibody that specifically binds to endothelial lipase has a third heavy chain complementarity determining region (CDR3) with an amino acid sequence at least 90% identical to SEQ ID NO: 3. In some embodiments of the pharmaceutical formulation, the antibody that specifically binds to endothelial lipase has a heavy chain CDR3 comprising SEQ ID NO:3.

In some embodiments of the pharmaceutical formulation, the antibody that specifically binds to endothelial lipase has a first light chain complementarity determining region (CDR1) with an amino acid sequence at least 90% identical to SEQ ID NO: 5. In some embodiments of the pharmaceutical formulation, the antibody that specifically binds to endothelial lipase has a light chain CDR1 comprising SEQ ID NO:5. In some embodiments of the pharmaceutical formulation, the antibody that specifically binds to endothelial lipase has a second light chain complementarity determining region (CDR2) with an amino acid sequence at least 80% identical to SEQ ID NO: 6. In some embodiments of the pharmaceutical formulation, the antibody that specifically binds to endothelial lipase has a light chain CDR2 comprising SEQ ID NO:6. In some embodiments of the pharmaceutical formulation, the antibody that specifically binds to endothelial lipase has a third light chain complementarity determining region (CDR3) with an amino acid sequence at least 80% identical to SEQ ID NO: 7. In some embodiments of the pharmaceutical formulation, the antibody that specifically binds to endothelial lipase has a light chain CDR3 comprising SEQ ID NO:7.

In some embodiments of the pharmaceutical formulation, the heavy chain of the antibody that specifically binds to endothelial lipase comprises an amino acid sequence at least 90%, at least 95%, at least 98% or at least 99% identical to SEQ ID NO: 4. In some embodiments of the pharmaceutical formulation, the light chain of the antibody that specifically binds to endothelial lipase comprises an amino acid sequence at least 90%, at least 95%, at least 98% or at least 99% identical to SEQ ID NO: 8. In some embodiments of the pharmaceutical formulation, the antibody that specifically binds to endothelial lipase comprises a heavy chain having an amino acid sequence comprising SEQ ID NO:4 and a light chain comprising an amino acid sequence comprising SEQ ID NO: 8.

In some embodiments of the pharmaceutical formulation, the antibody that specifically binds to endothelial lipase comprises a heavy chain comprising an amino acid sequence at least 90% identical to SEQ ID NO:9 and a light chain comprising an amino acid sequence at least 90% identical to SEQ ID NO:10. In some embodiments of the pharmaceutical formulation, the antibody that specifically binds to endothelial lipase comprises a heavy chain comprising SEQ ID NO:9 and a light chain comprising SEQ ID NO:10.

In some embodiments, the pharmaceutical formulation further comprises histidine. In some embodiments, the pharmaceutical formulation further comprises from about 5 mM to about 40 mM histidine. In some embodiments, the pharmaceutical formulation further comprises about 20 mM histidine. In some embodiments, the pharmaceutical formulation further comprises about 10 mM histidine. In some embodiments of the pharmaceutical formulation further comprising histidine, the histidine is a hydrochloride salt.

In some embodiments, the pharmaceutical formulation further comprises a surfactant. In some embodiments, the pharmaceutical formulation further comprises the surfactant polysorbate 20, polysorbate 60 or polysorbate 80. In some embodiments, the pharmaceutical formulation further comprises the surfactant polysorbate 80. In some embodiments, the pharmaceutical formulation further comprises from about 0.005% to about 0.05% polysorbate 80. In some embodiments, the pharmaceutical formulation further comprises about 0.01% polysorbate 80. In some embodiments, the pharmaceutical formulation further comprises about 0.02% polysorbate 80. In some embodiments, the pharmaceutical formulation further comprises about 0.04% polysorbate 80.

In some embodiments, the pharmaceutical formulation has a pH less than 7.0. In some embodiments, the pharmaceutical formulation has a pH of about 5.0 to about 7.0. In some embodiments, the pharmaceutical formulation has a pH of about 6.0. In some embodiments, the pharmaceutical formulation has a viscosity of less than 30 cP at 18° C. In some embodiments, the pharmaceutical formulation has an osmolality of about 260 to about 500 mOsm/kg.

In some embodiments, the pharmaceutical formulation is suitable for injection. In some embodiments, the pharmaceutical formulation is suitable for intravenous injection. In some embodiments, the pharmaceutical formulation is suitable for subcutaneous injection. In some embodiments, the pharmaceutical formulation is suitable for intramuscular injection.

In some embodiments, the pharmaceutical formulation is reconstituted from a lyophilized formulation. In some embodiments, the pharmaceutical formulation is a liquid formulation. In some embodiments, the pharmaceutical formulation is suitable for administration with an injection device such as an autoinjector device or a large bolus injector. In some embodiments, the pharmaceutical formulation is suitable for administration with an injection device such as a pre-filled syringe.

The present disclosure is also directed to methods comprising administering any of the pharmaceutical formulations disclosed herein. In some embodiments, the disclosure is directed to a method for treating cardiovascular disease in a mammalian subject in need thereof comprising administering to the subject any of the pharmaceutical formulations disclosed herein. In some embodiments, the disclosure is directed to a method for preventing cardiovascular disease in a mammalian subject in need thereof comprising administering to the subject any of the pharmaceutical formulations disclosed herein. In some embodiments, the disclosure is directed to a method for reducing atherosclerosis in a mammalian subject in need thereof comprising administering to the subject any of the pharmaceutical formulations disclosed herein. In some embodiments, the disclosure is directed to a method for reducing the risk of cardiovascular death, myocardial infarction, stroke, and/or coronary revascularization in a mammalian subject with prior acute coronary syndrome comprising administering to the subject any of the pharmaceutical formulations disclosed herein.

In some embodiments, the disclosure is directed to a method for preventing a secondary cardiovascular event in a mammalian subject in need thereof comprising administering to the subject any of the pharmaceutical formulations disclosed herein. In some embodiments, the disclosure is directed to a method for reducing the risk of a major adverse cardiovascular event in a mammalian subject in need thereof comprising administering to the subject any of the pharmaceutical formulations disclosed herein. In some embodiments, the disclosure is directed to a method for increasing the concentration of high-density lipoprotein in the blood of a mammalian subject in need thereof comprising administering to the subject any of the pharmaceutical formulations disclosed herein.

In some embodiments, the disclosure is directed to a method for increasing one or more clinical endpoints related to high-density lipoprotein in a mammalian subject in need thereof comprising administering to the subject any of the pharmaceutical formulations disclosed herein, wherein the clinical endpoints are HDL-c blood concentration, HDL particle number, HDL particle size, HDL phospholipid blood concentration, ApoA1 blood concentration and cholesterol efflux capacity. In some embodiments, the disclosure is directed to a method for decreasing inflammation in a mammalian subject in need thereof comprising administering to the subject any of the pharmaceutical formulations disclosed herein. In some embodiments, the disclosure is directed to a method for preventing inflammation in a mammalian subject in need thereof comprising administering to the subject any of the pharmaceutical formulations disclosed herein.

In embodiments of the methods disclosed herein, the pharmaceutical formulation is administered with an injection device. In embodiments of the methods disclosed herein, the pharmaceutical formulation is administered with an autoinjection device. In embodiments of the methods disclosed herein, the pharmaceutical formulation is administered with a large volume bolus injector. In embodiments of the methods disclosed herein, the pharmaceutical formulation is administered with a pre-filled syringe.

In embodiments of the methods disclosed herein, the mammalian subject is a human.

The present disclosure is further directed to a lyophilized pharmaceutical formulation comprising i) an antibody that specifically binds to endothelial lipase and ii) arginine hydrochloride; wherein the lyophilized pharmaceutical formulation can be reconstituted to a concentration of about 100 mg/mL to about 160 mg/mL of an antibody that specifically binds to endothelial lipase, and about 120 mM to about 260 mM arginine salt; and wherein the lyophilized pharmaceutical formulation is pharmaceutically acceptable.

The lyophilized pharmaceutical formulation comprising i) an antibody that specifically binds to endothelial lipase, ii) arginine hydrochloride, iii) histidine or a salt thereof, and iv) polysorbate 80; wherein the lyophilized pharmaceutical formulation can be reconstituted to a concentration of about 125 mg/mL antibody that specifically binds to endothelial lipase, about 160 mM arginine hydrochloride, about 20 mM histidine or a salt thereof and about 0.04% polysorbate 80; and wherein the lyophilized pharmaceutical formulation is pharmaceutically acceptable.

The present disclosure is further directed to a lyophilized pharmaceutical formulation comprising i) an antibody that specifically binds to endothelial lipase, ii) arginine hydrochloride, iii) histidine or a salt thereof, and iv) polysorbate 80; wherein the lyophilized pharmaceutical formulation can be reconstituted to a concentration of about 125 mg/mL antibody that specifically binds to endothelial lipase, about 220 mM arginine hydrochloride, about 20 mM histidine or a salt thereof and about 0.04% polysorbate 80; and wherein the lyophilized pharmaceutical formulation is pharmaceutically acceptable.

The present disclosure is further directed to a lyophilized pharmaceutical formulation comprising i) an antibody that specifically binds to endothelial lipase, ii) arginine hydrochloride, iii) histidine or a salt thereof, and iv) polysorbate 80; wherein the lyophilized pharmaceutical formulation can be reconstituted to a concentration of about 100 mg/mL antibody that specifically binds to endothelial lipase, about 160 mM arginine hydrochloride, about 20 mM histidine or a salt thereof and about 0.04% polysorbate 80; and wherein the lyophilized pharmaceutical formulation is pharmaceutically acceptable. In embodiments of the lyophilized pharmaceutical formulations disclosed herein, the lyophilized pharmaceutical formulation can be reconstituted to a pH of about 6.0.

The present disclosure is further directed to a liquid pharmaceutical formulation comprising about 125 mg/mL of an antibody that specifically binds to endothelial lipase, about 220 mM arginine hydrochloride, about 10 mM histidine or a salt thereof and about 0.02% polysorbate 80 and is pharmaceutically acceptable.

The present disclosure is further directed to a liquid pharmaceutical formulation comprising about 100 mg/mL of an antibody that specifically binds to endothelial lipase, about 220 mM arginine hydrochloride, about 10 mM histidine or a salt thereof and about 0.02% polysorbate 80 and is pharmaceutically acceptable.

The present disclosure is further directed to a liquid pharmaceutical formulation comprising about 125 mg/mL of an antibody that specifically binds to endothelial lipase, about 220 mM arginine hydrochloride, about 20 mM histidine or a salt thereof and about 0.02% polysorbate 80 and is pharmaceutically acceptable.

The present disclosure is further directed to a liquid pharmaceutical formulation comprising about 100 mg/mL of an antibody that specifically binds to endothelial lipase, about 220 mM arginine hydrochloride, about 20 mM histidine or a salt thereof and about 0.02% polysorbate 80 and is pharmaceutically acceptable.

The present disclosure is further directed to a liquid pharmaceutical formulation comprising about 50 mg/mL of an antibody that specifically binds to endothelial lipase, about 80 mM arginine hydrochloride, about 10 mM histidine or a salt thereof and about 0.02% polysorbate 80, and is pharmaceutically acceptable.

In embodiments of the liquid pharmaceutical formulations disclosed herein, the liquid pharmaceutical formulation has a pH of about 6.0.

### BRIEF DESCRIPTION OF DRAWINGS

FIG. 1A is plot of hydrodynamic radius vs. antibody concentration for results obtained from dynamic light scattering analysis of MEDI5884 formulations in either histidine and sucrose (squares); histidine, sucrose and arginine (diamonds); or histidine and arginine (triangles). FIG. 1B is a plot of the diffusion interaction parameter (Kd) values for each of these three formulations as determined by dynamic light scattering.

FIGS. 2A-C are plots of analytical ultracentrifugation analysis of the three formulations provided in FIG. 1. Sedimentation profiles, g(s), and weight average sedimentation coefficients, s, were obtained for MEDI5884 over the concentration range of 0.2-2.4 mg/mL. The results showed that the g(s) profiles significantly shifted toward higher s values from 4.6 s at 0.2 mg/mL to 4.9 s at 2.4 mg/mL, indicating that MEDI5884 undergoes self-association under the solution conditions examined (FIG. 2A). Such shifts are not observed for solution conditions under which the antibody does not self-associate (FIGS. 2B and 2C) indicated by overlapping g(s) peaks (i.e., peaks do not shift to larger s values as a function of increasing protein concentration).

FIGS. 3A and 3B are bar charts of viscosity in cP for buffer formulations of MEDI5884 at different pH values as shown. Formulations tested contained either 100 mg/mL (FIG. 3A) or 165 mg/mL (FIG. 3B) MEDI5884 in 160 mM ArgHCl and 20 mm of the buffer indicated at the pH indicated. Viscosity was measured at 18° C.

FIG. 4 is a bar chart of viscosity in cP for formulations of MEDI5884 having different excipients. The tested formulations contained 150 mg/mL MEDI5884, 20 mM His/His-HCl and the concentration of excipient shown at pH 6.0. Viscosity was measured at 18° C. The target viscosity is represented by a horizontal line across the bars.

FIG. 5 is a plot of viscosity in cP versus MEDI5884 concentration in mg/mL for three different concentrations of arginine: 190 mM (triangles), 220 mM (diamonds) and 250 mM (x's). The formulations also contained MEDI5884 at 145 mg/mL, 165 mg/mL or 175 mg/mL in 20 mM His/HisHCl 0.02% PS80. Viscosity was measured at 18° C.

FIG. 6 is a bar chart of viscosity in cP for formulations of MEDI5884 at varying pH values. The tested formulations contained 165 mg/mL MEDI5884 in 20 mM His/HisHCl and 220 mM ArgHCl and in either histidine or acetate buffer at the indicated pH values. Viscosity was measured at 18° C.

### DETAILED DESCRIPTION OF THE INVENTION

The present disclosure provides pharmaceutical formulations comprising an antibody that specifically binds to human endothelial lipase and an arginine salt. The present disclosure also provides methods for using the pharmaceutical formulations disclosed herein.

It should be appreciated that the particular implementations shown and described herein are examples and are not intended to otherwise limit the scope of the application in any way.

The published patents, patent applications, websites, company names, and scientific literature referred to herein are hereby incorporated by reference in their entirety to the same extent as if each was specifically and individually indicated to be incorporated by reference. Any conflict from any reference cited herein and the specific teachings of this specification shall be resolved in favor of the latter. Likewise, any conflict from an art-understood definition of a word or phrase and a definition of the word or phrase as specifically taught in this specification shall be resolved in favor of the latter.

As used herein, “a” or “an” may mean one or more. As used herein, when used in conjunction with the word “comprising,” the words “a” or “an” may mean one or more than one. As used herein, “another” or “a further” may mean at least a second or more.

Throughout this application, the term “about” is used to indicate that a value includes the inherent variation of error for the method/device being employed to determine the value, or the variation that exists among the study subjects. Typically, the term “about” is meant to encompass approximately or less than 1%, 2%, 3%, 4%, 5%, 6%, 7% 8%, 9%, 10%, 11%, 12%, 13%, 14%, 15%, 16%, 17%, 18%, 19% or 20% variability, depending on the situation.

The use of the term “or” in the claims is used to mean “and/or”, unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps. It is contemplated that any embodiment discussed in this specification can be implemented with respect to any method, system, host cells, expression vectors, and/or composition of the present disclosure. Furthermore, compositions, systems, host cells, and/or vectors of the present disclosure can be used to achieve methods and proteins of the present disclosure.

The use of the term “for example” and its corresponding abbreviation “e.g.” (whether italicized or not) means that the specific terms recited are representative examples and embodiments of the disclosure that are not intended to be limited to the specific examples referenced or cited unless explicitly stated otherwise.

Technical and scientific terms used herein have the meaning commonly understood by one of skill in the art to which the present application pertains, unless otherwise defined. Reference is made herein to various methodologies and materials known to those of skill in the art.

The term “antibody” as used herein can refer to a protein comprising at least two heavy chains and two light chains connected by disulfide bonds. The term “antibody” can include naturally occurring antibodies as well as all recombinant forms of antibodies, e.g., humanized antibodies, fully human antibodies and chimeric antibodies. Each heavy chain can comprise a heavy chain variable region (VH) and a heavy chain constant region (CH). Each light chain can comprise a light chain variable region (VL) and a light chain constant region (CL). The term “antibody”, however, can also include other types of antibodies such as single domain antibodies, heavy chain antibodies, i.e. antibodies only composed of one or more, in particular two heavy chains, and nanobodies, i.e. antibodies only composed of a single monomeric variable domain. Examples of fragments or derivatives of an antibody include (i) Fab fragments, monovalent fragments consisting of the variable region and the first constant domain of each the heavy and the light chain; (ii) F(ab)2 fragments, bivalent fragments comprising two Fab fragments linked by a disulfide bridge at the hinge region; (iii) Fd fragments consisting of the variable region and the first constant domain CHi of the heavy chain; (iv) Fv fragments consisting of the heavy chain and light chain variable region of a single arm of an antibody; (v) scFv fragments, Fv fragments consisting of a single polypeptide chain; (vi) (Fv)2 fragments consisting of two Fv fragments covalently linked together; (vii) a heavy chain variable domain; and (viii) multibodies consisting of a heavy chain variable region and a light chain variable region covalently linked together in such a manner that association of the heavy chain and light chain variable regions can only occur intermolecular but not intramolecular.

### Antibodies to Endothelial Lipase

Endothelial lipase (sometimes abbreviated LIPG or EL) is a lipase enzyme that is secreted by vascular endothelial cells. Endothelial lipase plays a role in lipoprotein metabolism, cytokine expression and the regulation of lipid composition in cells. Yu, et al., Histology and Histopathology 33 (1): 1-10 (2018). Endotheial lipase is a phospholipase of the triglyceride lipase family that catalyzes the conversion of high-density lipoprotein (HDL) to low-density lipoprotein (LDL). Jaye et al., Nature Genetics 21:424-428 (1999). Endothelial lipase is thought to play a role in cardiovascular diseases such as atherosclerosis. Paradis, et al., The Canadian Journal of Cardiology. 22 Suppl B (Suppl B): 31B-34B (2006).

The amino acid sequences for human endothelial lipase is known in the art and the mature version of the protein (lacking the leader sequence) is provided herein as the sequence of SEQ ID NO:13.

Mature Human Endothelial Lipase (lacking leader
sequence):
(SEQ ID NO: 13)
SPVPFGPEGRLEDKLHKPKATQTEVKPSVRFNLRTSKDPEHEGCYLSVG
HSQPLEDCSFNMTAKTFFIIHGWTMSGIFENWLHKLVSALHTREKDANV
VVVDWLPLAHQLYTDAVNNTRVVGHSIARMLDWLQEKDDFSLGNVHLIG
YSLGAHVAGYAGNFVKGTVGRITGLDPAGPMFEGADIHKRLSPDDADFV
DVLHTYTRSFGLSIGIQMPVGHIDIYPNGGDFQPGCGLNDVLGSIAYGT
ITEVVKCEHERAVHLFVDSLVNQDKPSFAFQCTDSNRFKKGICLSCRKN
RCNSIGYNAKKMRNKRNSKMYLKTRAGMPFRVYHYQMKIHVFSYKNMGE
IEPTFYVTLYGTNADSQTLPLEIVERIEQNATNTFLVYTEEDLGDLLKI
QLTWEGASQSWYNLWKEFRSYLSQPRNPGRELNIRRIRVKSGETQRKLT
FCTEDPENTSISPGRELWFRKCRDGWRMKNETSPTVELP

The pharmaceutical formulations disclosed comprise an antibody that specifically binds to endothelial lipase. In embodiments, exemplary antibodies that may be used in the pharmaceutical formulations are those disclosed in US Published Patent Application 2017/0260290 to Naito et al., which is hereby incorporated by reference herein for its disclosure of these antibodies. In embodiments, the antibody that specifically binds endothelial lipase has the heavy chain amino acid sequence from antibody h55A1-S6 disclosed in US2017/0260290. In embodiments, the antibody that specifically binds endothelial lipase has the light chain amino acid sequence from antibody h55A1-F1 disclosed in US2017/0260290. In embodiments, the antibody that specifically binds endothelial lipase has the heavy chain amino acid sequence from antibody h55A1-S6 and the light chain amino acid sequence from antibody h55A1-F1 as disclosed in US2017/0260290. Specific antibody sequences referred to herein are taken from US2017/0260290 as indicated in the Sequence table below.

In embodiments, the antibody that specifically binds to endothelial lipase is MEDI5884, as described herein and in the examples below.

In embodiments, the antibody that specifically binds to endothelial lipase comprises the six CDRs of the MEDI5884 antibody listed as provided in Tables 1 and 2.

TABLE 1
VH CDR Amino Acid Sequences¹

| Anti-body | VH CDR1 | VH CDR2 | VH CDR3 |
|---|---|---|---|
| MEDI5884 | NYALN (SEQ | WINTYSGVGTYAGEFKG | RGFYGRRFFDV |
| | ID NO: 1) | (SEQ ID NO: 2) | (SEQ ID NO: 3) |

¹The VH CDRs in Table 1 are determined according to Kabat.

TABLE 2
VL CDR Amino Acid Sequences²

| Antibody | VL CDR1 | VL CDR2 | VL CDR3 |
|---|---|---|---|
| MEDI5884 | KASQSVDYDVDSYMH | AASNLAS (SEQ ID | QQTIEDPPT |
| | (SEQ ID NO: 5) | NO: 6) | (SEQ ID NO: 7) |

²The VL CDRs in Table 2 are determined according to Kabat.

In embodiments, the antibody that specifically binds to endothelial lipase comprises the VH of the MEDI5884 antibody listed in Table 3.

TABLE 3
Variable Heavy Chain (VH) Amino Acid Sequences

| Antibody | VH Amino Acid Sequence |
|---|---|
| MEDI5884 | QVQLVQSGSELKKPGASVKVSCKASGYTFTNYALNWVRQAPGQGLE |
| | WMGWINTYSGVGTYAGEFKGRFVFSLDTSVSTAYLQISSLKAEDTAV |
| | YYCARRGFYGRRFFDVWGKGTTVTVSS (SEQ ID NO: 4) |

In embodiments, the antibody that specifically binds to endothelial lipase comprises the VL of the MEDI5884 antibody listed in Table 4.

TABLE 4
Variable Light Chain (VL) Amino Acid Sequences

| Antibody | VL Amino Acid Sequence |
|---|---|
| MEDI5884 | DIQLTQSPSSLSASVGDRVTITCKASQSVDYDVDSYMHWYQQKPGK |
| | APKLLIYAASNLASGVPSRFSGSGSGTDFTFTISSLQPEDIATYYCQQTI |
| | EDPPTFGGGTKVEIK (SEQ ID NO: 8) |

In embodiments, the antibody that specifically binds to endothelial lipase comprises the VH and the VL of the MEDI5884 antibody listed in Tables 3 and 4.

In embodiments, the antibody that specifically binds to endothelial lipase comprises the heavy chain sequence of the MEDI5884 antibody listed in Table 5.

TABLE 5
Full-length heavy chain amino acid sequences

| Antibody | Full-Length Heavy Chain Amino Acid Sequence (SEQ ID NO: 9) |
|---|---|
| MEDI5884 | QVQLVQSGSELKKPGASVKVSCKASGYTFTNYALNWVRQAPGQGL |
| | EWMGWINTYSGVGTYAGEFKGRFVFSLDTSVSTAYLQISSLKAEDT |
| | AVYYCARRGFYGRRFFDVWGKGTTVTVSSASTKGPSVFPLAPCSRS |
| | TSESTAALGCLVKDYFPEPVTVSWNSGALTSGVHTFPAVLQSSGLYS |
| | LSSVVTVPSSSLGTKTYTCNVDHKPSNTKVDKRVESKYGPPCPPCPA |
| | PEFLGGPSVFLFPPKPKDTLMISRTPEVTCVVVDVSQEDPEVQFNWY |
| | VDGVEVHNAKTKPREEQFNSTYRVVSVLTVLHQDWLNGKEYKCKV |
| | SNKGLPSSIEKTISKAKGQPREPQVYTLPPSQEEMTKNQVSLTCLVK |
| | GFYPSDIAVEWESNGQPENNYKTTPPVLDSDGSFFLYSRLTVDKSRW |
| | QEGNVFSCSVMHEALHNHYTQKSLSLSLGK (SEQ ID NO: 9) |

In embodiments, the antibody that specifically binds to endothelial lipase comprises the light chain sequence of the MEDI5884 antibody listed in Table 6.

TABLE 6
Full-length light chain amino acid sequences

| Antibody | Full-Length Light Chain Amino Acid Sequence (SEQ ID NO: 10) |
|---|---|
| MEDI5884 | DIQLTQSPSSLSASVGDRVTITCKASQSVDYDVDSYMHWYQQKPGK |
| | APKLLIYAASNLASGVPSRFSGSGSGTDFTFTISSLQPEDIATYYCQQT |
| | IEDPPTFGGGTKVEIKRTVAAPSVFIFPPSDEQLKSGTASVVCLLNNF |
| | YPREAKVQWKVDNALQSGNSQESVTEQDSKDSTYSLSSTLTLSKAD |
| | YEKHKVYACEVTHQGLSSPVTKSFNRGEC (SEQ ID NO: 10) |

In embodiments, the antibody that specifically binds to endothelial lipase comprises the heavy chain sequence and the light chain sequence of the MEDI5884 antibody listed in Tables 5 and 6.

With respect to the heavy chain of the antibody that binds endothelial lipase, in embodiments, the heavy chain is a gamma heavy chain. The constant region of a human IgG4P heavy chain can comprise the following amino acid sequence:

(SEQ ID NO: 11)
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPPCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK.

With respect to the light chain of the antibody that binds endothelial lipase, in some aspects of the present disclosure, the light chain is a kappa light chain. The constant region of a human C kappa light chain can comprise the following amino acid sequence:

(SEQ ID NO: 12)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC.

In embodiments, the antibody that specifically binds endothelial lipase has a first heavy chain complementarity determining region (CDR1) with an amino acid sequence at least 80% identical to SEQ ID NO: 1. In embodiments, the antibody that specifically binds endothelial lipase has a second heavy chain complementarity determining region (CDR2) with an amino acid sequence at least 80% identical to SEQ ID NO: 2. In embodiments, the antibody that specifically binds endothelial lipase has a third heavy chain complementarity determining region (CDR3) with an amino acid sequence at least 80% identical to SEQ ID NO: 3. In embodiments, the antibody that specifically binds endothelial lipase has two heavy chain complementarity determining regions at least 80% identical to two of SEQ ID NOs. 1-3. In embodiments, the antibody that specifically binds endothelial lipase has heavy chain complementarity determining regions at least 80% identical to each of SEQ ID NOs. 1-3. In embodiments, the antibody that specifically binds endothelial lipase has a heavy chain variable domain amino acid sequence at least 80% identical to SEQ ID NO: 4. In embodiments, the antibody that specifically binds endothelial lipase has a heavy chain amino acid sequence at least 80% identical to SEQ ID NO: 9.

In embodiments, the antibody that specifically binds endothelial lipase has a first heavy chain complementarity determining region (CDR1) with an amino acid sequence at least 90% identical to SEQ ID NO: 1. In embodiments, the antibody that specifically binds endothelial lipase has a second heavy chain complementarity determining region (CDR2) with an amino acid sequence at least 90% identical to SEQ ID NO: 2. In embodiments, the antibody that specifically binds endothelial lipase has a third heavy chain complementarity determining region (CDR3) with an amino acid sequence at least 90% identical to SEQ ID NO: 3. In embodiments, the antibody that specifically binds endothelial lipase has two heavy chain complementarity determining regions at least 90% identical to two of SEQ ID NOs. 1-3. In embodiments, the antibody that specifically binds endothelial lipase has heavy chain complementarity determining regions at least 90% identical to each of SEQ ID NOs. 1-3. In embodiments, the antibody that specifically binds endothelial lipase has a heavy chain variable domain amino acid sequence at least 90% identical to SEQ ID NO: 4. In embodiments, the antibody that specifically binds endothelial lipase has a heavy chain amino acid sequence at least 90% identical to SEQ ID NO: 9.

In embodiments, the antibody that specifically binds endothelial lipase has a heavy chain CDR1 with an amino acid sequence at least 95% identical to SEQ ID NO: 1. In embodiments, the antibody that specifically binds endothelial lipase has a heavy chain CDR2 with an amino acid sequence at least 95% identical to SEQ ID NO: 2. In embodiments, the antibody that specifically binds endothelial lipase has a heavy chain CDR3 with an amino acid sequence at least 95% identical to SEQ ID NO: 3. In embodiments, the antibody that specifically binds endothelial lipase has two heavy chain CDRs at least 95% identical to two of SEQ ID NOs. 1-3. In embodiments, the antibody that specifically binds endothelial lipase has heavy chain CDRs at least 95% identical to each of SEQ ID NOs. 1-3. In embodiments, the antibody that specifically binds endothelial lipase has a heavy chain variable domain amino acid sequence at least 95% identical to SEQ ID NO: 4. In embodiments, the antibody that specifically binds endothelial lipase has a heavy chain amino acid sequence at least 95% identical to SEQ ID NO: 9.

In embodiments, the antibody that specifically binds endothelial lipase has a heavy chain CDR1 with an amino acid sequence at least 98% identical to SEQ ID NO: 1. In embodiments, the antibody that specifically binds endothelial lipase has a heavy chain CDR2 with an amino acid sequence at least 98% identical to SEQ ID NO: 2. In embodiments, the antibody that specifically binds endothelial lipase has a heavy chain CDR3 with an amino acid sequence at least 98% identical to SEQ ID NO: 3. In embodiments, the antibody that specifically binds endothelial lipase has two heavy chain CDRs at least 98% identical to two of SEQ ID NOs. 1-3. In embodiments, the antibody that specifically binds endothelial lipase has heavy chain CDRs at least 98% identical to each of SEQ ID NOs. 1-3. In embodiments, the antibody that specifically binds endothelial lipase has a heavy chain variable domain amino acid sequence at least 98% identical to SEQ ID NO: 4. In embodiments, the antibody that specifically binds endothelial lipase has a heavy chain amino acid sequence at least 98% identical to SEQ ID NO: 9.

In embodiments, the antibody that specifically binds endothelial lipase has a heavy chain CDR1 with an amino acid sequence at least 99% identical to SEQ ID NO: 1. In embodiments, the antibody that specifically binds endothelial lipase has a heavy chain CDR2 with an amino acid sequence at least 99% identical to SEQ ID NO: 2. In embodiments, the antibody that specifically binds endothelial lipase has a heavy chain CDR3 with an amino acid sequence at least 99% identical to SEQ ID NO: 3. In embodiments, the antibody that specifically binds endothelial lipase has two heavy chain CDRs at least 99% identical to two of SEQ ID NOs. 1-3. In embodiments, the antibody that specifically binds endothelial lipase has heavy chain CDRs at least 99% identical to each of SEQ ID NOs. 1-3. In embodiments, the antibody that specifically binds endothelial lipase has a heavy chain variable domain amino acid sequence at least 99% identical to SEQ ID NO: 4. In embodiments, the antibody that specifically binds endothelial lipase has a heavy chain amino acid sequence at least 99% identical to SEQ ID NO: 9.

In embodiments, the antibody that specifically binds endothelial lipase has a heavy chain CDR1 with an amino acid sequence comprising SEQ ID NO: 1. In embodiments, the antibody that specifically binds endothelial lipase has a heavy chain CDR2 with an amino acid sequence comprising SEQ ID NO: 2. In embodiments, the antibody that specifically binds endothelial lipase has a heavy chain CDR3 with an amino acid sequence comprising SEQ ID NO: 3. In embodiments, the antibody that specifically binds endothelial lipase has two heavy chain CDRs comprising two of SEQ ID NOs. 1-3. In embodiments, the antibody that specifically binds endothelial lipase has heavy chain CDRs comprising each of SEQ ID NOs. 1-3. In embodiments, the antibody that specifically binds endothelial lipase has a heavy chain variable domain amino acid sequence comprising SEQ ID NO: 4. In embodiments, the antibody that specifically binds endothelial lipase has a heavy chain amino acid sequence comprising SEQ ID NO: 9.

In embodiments, the antibody that specifically binds endothelial lipase has a light chain CDR1 with an amino acid sequence at least 80% identical to SEQ ID NO: 5. In embodiments, the antibody that specifically binds endothelial lipase has a light chain CDR2 with an amino acid sequence at least 80% identical to SEQ ID NO: 6. In embodiments, the antibody that specifically binds endothelial lipase has a light chain CDR3 with an amino acid sequence at least 80% identical to SEQ ID NO: 7. In embodiments, the antibody that specifically binds endothelial lipase has two light chain CDRs at least 80% identical to two of SEQ ID NOs. 5-7. In embodiments, the antibody that specifically binds endothelial lipase has light chain CDRs at least 80% identical to each of SEQ ID NOs. 5-7. In embodiments, the antibody that specifically binds endothelial lipase has a light chain variable domain amino acid sequence at least 80% identical to SEQ ID NO: 8. In embodiments, the antibody that specifically binds endothelial lipase has a light chain amino acid sequence at least 80% identical to SEQ ID NO: 10.

In embodiments, the antibody that specifically binds endothelial lipase has a light chain CDR1 with an amino acid sequence at least 90% identical to SEQ ID NO: 5. In embodiments, the antibody that specifically binds endothelial lipase has a light chain CDR2 with an amino acid sequence at least 90% identical to SEQ ID NO: 6. In embodiments, the antibody that specifically binds endothelial lipase has a light chain CDR3 with an amino acid sequence at least 90% identical to SEQ ID NO: 7. In embodiments, the antibody that specifically binds endothelial lipase has two light chain CDRs at least 90% identical to two of SEQ ID NOs. 5-7. In embodiments, the antibody that specifically binds endothelial lipase has light chain CDRs at least 90% identical to each of SEQ ID NOs. 5-7. In embodiments, the antibody that specifically binds endothelial lipase has a light chain variable domain amino acid sequence at least 90% identical to SEQ ID NO: 8. In embodiments, the antibody that specifically binds endothelial lipase has a light chain amino acid sequence at least 90% identical to SEQ ID NO: 10.

In embodiments, the antibody that specifically binds endothelial lipase has a light chain CDR1 with an amino acid sequence at least 95% identical to SEQ ID NO: 5. In embodiments, the antibody that specifically binds endothelial lipase has a light chain CDR2 with an amino acid sequence at least 95% identical to SEQ ID NO: 6. In embodiments, the antibody that specifically binds endothelial lipase has a light chain CDR3 with an amino acid sequence at least 95% identical to SEQ ID NO: 7. In embodiments, the antibody that specifically binds endothelial lipase has two light chain CDRs at least 95% identical to two of SEQ ID NOs. 5-7. In embodiments, the antibody that specifically binds endothelial lipase has light chain CDRs at least 95% identical to each of SEQ ID NOs. 5-7. In embodiments, the antibody that specifically binds endothelial lipase has a light chain variable domain amino acid sequence at least 95% identical to SEQ ID NO: 8. In embodiments, the antibody that specifically binds endothelial lipase has a light chain amino acid sequence at least 95% identical to SEQ ID NO: 10.

In embodiments, the antibody that specifically binds endothelial lipase has a light chain CDR1 with an amino acid sequence at least 98% identical to SEQ ID NO: 5. In embodiments, the antibody that specifically binds endothelial lipase has a light chain CDR2 with an amino acid sequence at least 98% identical to SEQ ID NO: 6. In embodiments, the antibody that specifically binds endothelial lipase has a light chain CDR3 with an amino acid sequence at least 98% identical to SEQ ID NO: 7. In embodiments, the antibody that specifically binds endothelial lipase has two light chain CDRs at least 98% identical to two of SEQ ID NOs. 5-7. In embodiments, the antibody that specifically binds endothelial lipase has light chain CDRs at least 98% identical to each of SEQ ID NOs. 5-7. In embodiments, the antibody that specifically binds endothelial lipase has a light chain variable domain amino acid sequence at least 98% identical to SEQ ID NO: 8. In embodiments, the antibody that specifically binds endothelial lipase has a light chain amino acid sequence at least 98% identical to SEQ ID NO: 10.

In embodiments, the antibody that specifically binds endothelial lipase has a light chain CDR1 with an amino acid sequence at least 99% identical to SEQ ID NO: 5. In embodiments, the antibody that specifically binds endothelial lipase has a light chain CDR2 with an amino acid sequence at least 99% identical to SEQ ID NO: 6. In embodiments, the antibody that specifically binds endothelial lipase has a light chain CDR3 with an amino acid sequence at least 99% identical to SEQ ID NO: 7. In embodiments, the antibody that specifically binds endothelial lipase has two light chain CDRs at least 99% identical to two of SEQ ID NOs. 5-7. In embodiments, the antibody that specifically binds endothelial lipase has light chain CDRs at least 99% identical to each of SEQ ID NOs. 5-7. In embodiments, the antibody that specifically binds endothelial lipase has a light chain variable domain amino acid sequence at least 99% identical to SEQ ID NO: 8. In embodiments, the antibody that specifically binds endothelial lipase has a light chain amino acid sequence at least 99% identical to SEQ ID NO: 10.

In embodiments, the antibody that specifically binds endothelial lipase has a light chain CDR1 with an amino acid sequence comprising SEQ ID NO: 5. In embodiments, the antibody that specifically binds endothelial lipase has a light chain CDR2 with an amino acid sequence comprising SEQ ID NO: 6. In embodiments, the antibody that specifically binds endothelial lipase has a light chain CDR3 with an amino acid sequence comprising SEQ ID NO: 7. In embodiments, the antibody that specifically binds endothelial lipase has two light chain CDRs comprising two of SEQ ID NOs. 5-7. In embodiments, the antibody that specifically binds endothelial lipase has light chain CDRs comprising each of SEQ ID NOs. 5-7. In embodiments, the antibody that specifically binds endothelial lipase has a light chain variable domain amino acid sequence comprising SEQ ID NO: 8. In embodiments, the antibody that specifically binds endothelial lipase has a light chain amino acid sequence comprising SEQ ID NO: 10.

In embodiments, the antibody that specifically binds endothelial lipase comprises one of more complementarity determining regions selected from SEQ ID NOs: 1-3 and 5-7. In embodiments, the antibody that specifically binds endothelial lipase has a heavy chain variable domain amino acid sequence comprising SEQ ID NO: 4 and a light chain variable domain amino acid sequence comprising SEQ ID NO: 8.

In embodiments, the concentration of the antibody that specially binds endothelial lipase in the pharmaceutical formulation is from about 40 mg/mL and about 160 mg/mL. In embodiments, the concentration of the antibody in the pharmaceutical formulation is from about 100 mg/mL and about 150 mg/mL. In embodiments, the concentration of the antibody in the pharmaceutical formulation is from about 110 mg/mL and about 140 mg/mL. In embodiments, the concentration of the antibody in the pharmaceutical formulation is from about 120 mg/mL and about 130 mg/mL. In embodiments, the concentration of the antibody in the pharmaceutical formulation is from about 40 mg/mL and about 60 mg/mL. In embodiments, the concentration of the antibody in the pharmaceutical formulation is from about 90 mg/mL and about 110 mg/mL. In embodiments, the concentration of the antibody in the pharmaceutical formulation is about 125 mg/mL. In embodiments, the concentration of the antibody in the pharmaceutical formulation is about 50 mg/mL. In embodiments, the concentration of the antibody in the pharmaceutical formulation is about 100 mg/mL.

### Pharmaceutical Formulations

The pharmaceutical formulations disclosed provide a high enough concentration of antibody specific to endothelial lipase to be effective in a low volume suitable for injection. In embodiments, the pharmaceutical formulations provide a high enough concentration of antibody specific to endothelial lipase to be effective at a viscosity suitable for injection. In embodiments, the pharmaceutical formulations provide a high enough concentration of antibody specific to endothelial lipase to be effective while avoiding undesirable reversible self-association (RSA). In embodiments, the pharmaceutical formulations provide a high enough concentration of antibody specific to endothelial lipase to be effective at an osmolality suitable for injection. In embodiments, the pharmaceutical formulations provide a high enough concentration of antibody specific to endothelial lipase to be effective while being storage stable.

It is frequently a challenge when formulating injectable antibodies to be able to formulate the antibody at concentration high enough to allow for effective dosing in a volume suitable for injection. Increasing the concentration of antibody in a formulation can lead to undesirable increases in viscosity, RSA, precipitation and cloudiness that make the formulation unsuitable for injection into a subject.

The pharmaceutical formulations disclosed herein comprise an arginine (Arg) salt as a viscosity regulating agent. In embodiments, the arginine salt is arginine hydrochloride, arginine acetate, arginine glutamate or arginine sulfate. In embodiments, the arginine salt is arginine hydrochloride. In embodiments, the pharmaceutical formulations may comprise more than one arginine salt, for example, mixtures of two or more of arginine hydrochloride, arginine acetate, arginine glutamate and arginine sulfate

In embodiments, the concentration of the arginine salt in the pharmaceutical formulation is from about 50 mM to about 260 mM. In embodiments, the concentration of the arginine salt in the pharmaceutical formulation is from about 80 mM to about 260 mM. In embodiments, the concentration of the arginine salt in the pharmaceutical formulation is from about 160 mM to about 240 mM. In embodiments, the concentration of the arginine salt in the pharmaceutical formulation is from about 180 mM to about 230 mM. In embodiments, the concentration of the arginine salt in the pharmaceutical formulation is from about 200 mM to about 225 mM. In embodiments, the concentration of the arginine salt in the pharmaceutical formulation is from about 110 mM to about 140 mM. In embodiments, the concentration of the arginine salt in the pharmaceutical formulation is from about 50 mM to about 110 mM. In embodiments, the concentration of the arginine salt in the pharmaceutical formulation is from about 60 mM to about 100 mM. In embodiments, the concentration of the arginine salt in the pharmaceutical formulation is from about 70 mM to about 90 mM. In embodiments, the concentration of the arginine salt in the pharmaceutical formulation is about 220 mM. In embodiments, the concentration of the arginine salt in the pharmaceutical formulation is about 160 mM. In embodiments, the concentration of the arginine salt in the pharmaceutical formulation is about 80 mM.

In embodiments, the pharmaceutical formulation has a concentration of from about 50 mM to about 260 mM arginine hydrochloride (ArgHCl). In embodiments, the pharmaceutical formulation has a concentration of from about 80 mM to about 260 mM arginine hydrochloride. In embodiments, the pharmaceutical formulation has a concentration of from about 160 mM to about 240 mM arginine hydrochloride. In embodiments, the pharmaceutical formulation has a concentration of from about 180 mM to about 230 mM arginine hydrochloride. In embodiments, the pharmaceutical formulation has a concentration of from about 200 mM to about 225 mM arginine hydrochloride. In embodiments, the pharmaceutical formulation has a concentration of from about 110 mM to about 140 mM arginine hydrochloride (ArgHCl). In embodiments, the pharmaceutical formulation has a concentration of from about 50 mM to about 110 mM arginine hydrochloride. In embodiments, the pharmaceutical formulation has a concentration of from about 60 mM to about 100 mM arginine hydrochloride. In embodiments, the pharmaceutical formulation has a concentration of from about 70 mM to about 90 mM arginine hydrochloride. In embodiments, the pharmaceutical formulation has a concentration of about 220 mM arginine hydrochloride. In embodiments, the pharmaceutical formulation has a concentration of about 160 mM arginine hydrochloride. In embodiments, the pharmaceutical formulation has a concentration of about 80 mM arginine hydrochloride.

In embodiments, the pharmaceutical formulations further comprise histidine (His) as a buffer. In embodiments, the histidine in the pharmaceutical formulation may be in base form or salt form, or in combinations thereof. In embodiments where the histidine is in salt form, the salt form may be histidine hydrochloride, histidine acetate, histidine or histidine sulfate, or combinations thereof. In embodiments, the pharmaceutical formulation comprises histidine hydrochloride (HisHCl) In embodiments, the pharmaceutical formulation has a concentration of from about 5 mM to about 40 mM histidine and/or histidine salt. In embodiments, the pharmaceutical formulation has a concentration of about 10 mM histidine and/or histidine salt. In embodiments, the pharmaceutical formulation has a concentration of about 20 mM histidine and/or histidine salt.

In embodiments, the pharmaceutical formulations comprise other buffers such as acetate and phosphate buffers. In embodiments, the pharmaceutical formulation has a concentration of from about 5 mM to about 40 mM buffer. In embodiments, the pharmaceutical formulation has a concentration of about 10 mM buffer. In embodiments, the pharmaceutical formulation has a concentration of about 20 mM buffer.

In embodiments, the pharmaceutical formulations further comprise a surfactant. In embodiments, the surfactant is polysorbate 20, polysorbate 60, polysorbate 80 or combinations thereof. In embodiments, the surfactant is polysorbate 80 (PS80). In embodiments, the pharmaceutical formulation has a concentration of from about 0.005% to about 0.05% surfactant. In embodiments, the pharmaceutical formulation has a concentration of from about 0.02% to about 0.04% surfactant. In embodiments, the pharmaceutical formulation has a concentration of about 0.02% surfactant. In embodiments, the pharmaceutical formulation has a concentration of about 0.04% surfactant. In embodiments, the pharmaceutical formulation has a concentration of about 0.01% surfactant. In embodiments, the pharmaceutical formulation has a concentration of from about 0.01% to about 0.05% PS80. In embodiments, the pharmaceutical formulation has a concentration of from about 0.02% to about 0.04% PS80. In embodiments, the pharmaceutical formulation has a concentration of about 0.02% PS80. In embodiments, the pharmaceutical formulation has a concentration of about 0.04% PS80. In embodiments, the pharmaceutical formulation has a concentration of about 0.01% PS80.

In embodiments, the pharmaceutical formulations can further comprise additional excipients. In embodiments, the pharmaceutical formulation further comprises excipients suitable for use in injectable formulations, e.g., buffers, viscosity regulating agents, salts and stabilizers. In embodiments, the pharmaceutical formulation further comprises one or more of lysine, lysine hydrochloride, proline, glutamic acid, sodium sulfate, sodium chloride or dimethyl sulfoxide (DMSO).

In embodiments, the pharmaceutical formulations have a pH of less than 7.0. In embodiments, the pharmaceutical formulations have a pH of from about 5.0 to about 7.0. In embodiments, the pharmaceutical formulations have a pH of about 6.0.

The pharmaceutical formulations are designed to have a viscosity suitable for injection, e.g., with either an autoinjection of manual injection device. A formulation with too high of a viscosity requires greater injection force may not flow properly through the injection device either at all or only with the use of an undesirable large bore needle. Viscosity of such formulations is measured using methods known in the art as described further in the examples. As viscosity changes with temperature, it is usually measured at a common temperature, for example, 18° C. In embodiments, the pharmaceutical formulation has a viscosity of less than or equal to 30 centipoise (cP) at 18° C., i.e., about 1 to about 30 cP, about 2 to about 28 cP, about 3 to about 26 cP, about 5 to about 25 cP, about 6 to about 22 cP, about 7 to about 20 cP, about 8 to about 20 cP, about 9 to about 20 cP, about 10 to about 20 cP, or about 12 to about 18 cP at 18° C. In embodiments, the pharmaceutical formulation has a viscosity of less than or equal to 20 centipoise (cP) at 18° C. In embodiments, the pharmaceutical formulation has a viscosity of less than or equal to 15 centipoise (cP) at 18° C.

The pharmaceutical formulations are designed to have a volume suitable for injection with either an autoinjector or manual injector. In embodiments, the pharmaceutical formulation can have a volume of about 0.1 to about 4.0 mL, about 0.5 to about 3.5 mL, about 1.0 to about 3.0 mL, or about 1.5 to about 2.0 mL. In embodiments, the pharmaceutical formulation can have an injection volume of about 0.1 mL, about 0.25 mL, about 0.5 mL, about 0.75 mL, about 1.0 mL, about 1.25 mL, about 1.5 mL, about 1.75 mL, about 2.0 mL, about 2.25 mL, about 2.5, mL, about 2.75 mL, about 3.0 mL, about 3.25 mL, about 3.5 mL, about 3.75 mL or about 4.0 mL.

The pharmaceutical formulations can also be designed to prevent reversable self-association (RSA) between the antibody molecules in the formulation. RSA includes native, non-covalent, and reversible oligomerization of monomeric species of antibody or other protein. RSA is more likely in formulations having a high concentration of antibody because of the reduced intermolecular distance between individual molecules and the increased probability of molecular collisions. RSA can pose manufacturing and delivery challenges and can cause pain upon administration. RSA can be measured using techniques known in the art such as dynamic light scattering, analytical ultracentrifugation, differential scanning calorimetry, differential scanning fluorimetry and high performance size-exclusion chromatography.

The pharmaceutical formulations can also be designed to have an osmolality suitable for injection. The osmolality of an injectable formulation can be associated with pain upon injection and tolerability. Specific instruments and techniques for measuring osmolality are available in the art and described in the examples below. In embodiments, the pharmaceutical formulation has an osmolality of from about 260 mOsm/kg to about 500 mOsm/kg, i.e. from about 280 mOsm/kg to about 490 mOsm/kg, from about 320 mOsm/kg to about 480 mOsm/kg, from about 340 mOsm/kg to about 470 mOsm/kg, from about 360 mOsm/kg to about 460 mOsm/kg, from about 380 mOsm/kg to about 460 mOsm/kg, from about 400 mOsm/kg to about 460 mOsm/kg and from about 440 mOsm/kg to about 460 mOsm/kg. In embodiments, the pharmaceutical formulation has an osmolality of from about 440 mOsm/kg to about 460 mOsm/kg.

The pharmaceutical formulations can be designed to be suitable for injection to a subject and are suitable for use in injection systems known in the art. In embodiments, the pharmaceutical formulation is suitable for intravenous injection. In embodiments, the pharmaceutical formulation is suitable for subcutaneous injection. In embodiments, the pharmaceutical formulation is suitable for injection with an injection device. In embodiments, the pharmaceutical formulation is suitable for injection with an autoinjection device. In embodiments, the pharmaceutical formulation is suitable for injection with a large volume bolus injector. In embodiments, the pharmaceutical formulation is suitable for injection with a pre-filled syringe.

In embodiments, the pharmaceutical formulation is reconstituted from a lyophilized formulation. Liquids containing the formulation to be lyophilized can be lyophilized according to standard methods in the art. The lyophilized version of the formulation may be referred to as a drug product (DP) formulation. In embodiments, the lyophilized DP is reconstituted using the same volume of liquid present prior to lyophilization. In embodiments, the lyophilized DP is reconstituted using half the volume of liquid present prior to lyophilization (a half reconstitution or half recon). In a half reconstitution, the concentration of each component following reconstitution is then double the concentration of these components in the liquid prior to lyophilization. In embodiments, the lyophilized DP can be reconstituted with less volume than the original liquid. In embodiments, the lyophilized DP can be reconstituted with more volume than the original liquid.

In embodiments, the pharmaceutical formulation is a liquid formulation. The liquid version of the formulation may be referred to as a drug substance (DS) formulation or a liquid drug product (DP) formulation.

The pharmaceutical formulations can be designed to be storage stable. In embodiments, the pharmaceutical formulation is storage stable in lyophilized form. In embodiments, the pharmaceutical formulation is storage stable in liquid form. In embodiments, the pharmaceutical formulation is storage stable at a temperature of between about −80° C. to about −10° C. for a period of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 48 or 60 months. In embodiments, the pharmaceutical formulation is storage stable at a temperature of between about 16° C. to about 22° C. for a period of 1, 2, 3, 4, 5, 6, 7, 8, 9, 10, 11, 12, 13, 14, 15, 16, 17, or 18 months.

In embodiments, the pharmaceutical formulation is a lyophilized pharmaceutical formulation comprising an antibody that specifically binds to endothelial lipase and arginine hydrochloride, wherein the lyophilized pharmaceutical formulation can be reconstituted to a concentration of about 100 mg/mL to about 160 mg/mL of an antibody that specifically binds to endothelial lipase, and about 120 mM to about 260 mM arginine salt.

In embodiments, the pharmaceutical formulation is a lyophilized pharmaceutical formulation comprising an antibody that specifically binds to endothelial lipase, arginine hydrochloride, histidine or a salt thereof, and polysorbate 80, wherein the lyophilized pharmaceutical formulation can be reconstituted to a concentration of about 125 mg/mL antibody that specifically binds to endothelial lipase, about 160 mM arginine hydrochloride, about 20 mM histidine or a salt thereof and about 0.04% polysorbate 80.

In embodiments, the pharmaceutical formulation is a lyophilized pharmaceutical formulation comprising an antibody that specifically binds to endothelial lipase, arginine hydrochloride, histidine or a salt thereof, and polysorbate 80, wherein the lyophilized pharmaceutical formulation can be reconstituted to a concentration of about 125 mg/mL antibody that specifically binds to endothelial lipase, about 220 mM arginine hydrochloride, about 20 mM histidine or a salt thereof and about 0.04% polysorbate 80.

In embodiments, the pharmaceutical formulation is a lyophilized pharmaceutical formulation comprising an antibody that specifically binds to endothelial lipase, arginine hydrochloride, histidine or a salt thereof, and polysorbate 80, wherein the lyophilized pharmaceutical formulation can be reconstituted to a concentration of about 100 mg/mL antibody that specifically binds to endothelial lipase, about 160 mM arginine hydrochloride, about 20 mM histidine or a salt thereof and about 0.04% polysorbate 80.

In embodiments, the pharmaceutical formulation is a liquid pharmaceutical formulation comprising about 125 mg/mL of an antibody that specifically binds to endothelial lipase, about 220 mM arginine hydrochloride, about 10 mM histidine or a salt thereof and about 0.02% polysorbate 80.

In embodiments, the pharmaceutical formulation is a liquid pharmaceutical formulation comprising about 100 mg/mL of an antibody that specifically binds to endothelial lipase, about 220 mM arginine hydrochloride, about 10 mM histidine or a salt thereof and about 0.02% polysorbate 80.

In embodiments, the pharmaceutical formulation is a liquid pharmaceutical formulation comprising about 125 mg/mL of an antibody that specifically binds to endothelial lipase, about 220 mM arginine hydrochloride, about 20 mM histidine or a salt thereof and about 0.02% polysorbate 80.

In embodiments, the pharmaceutical formulation is a liquid pharmaceutical formulation comprising about 100 mg/mL of an antibody that specifically binds to endothelial lipase, about 220 mM arginine hydrochloride, about 20 mM histidine or a salt thereof and about 0.02% polysorbate 80.

In embodiments, the pharmaceutical formulation is a liquid pharmaceutical formulation comprising about 50 mg/mL of an antibody that specifically binds to endothelial lipase, about 80 mM arginine hydrochloride, about 10 mM histidine or a salt thereof and about 0.02% polysorbate 80.

### Methods

The present disclosure is also directed to methods comprising administering any of the pharmaceutical formulations disclosed herein. The methods described involve inhibition of a function of endothelial lipase by administration of a pharmaceutical formulation comprising an antibody that specifically binds to endothelial lipase. In embodiments, the methods comprise administering a pharmaceutical formulation comprising an antibody that specifically binds to endothelial lipase and an arginine salt.

In embodiments, the pharmaceutical formulation is used in a method for treating cardiovascular disease in a mammalian subject in need thereof. In embodiments, the pharmaceutical formulation is used in a method for preventing cardiovascular disease in a mammalian subject in need thereof. In embodiments, the pharmaceutical formulation is used in a method for reducing atherosclerosis in a mammalian subject in need thereof. In embodiments, the pharmaceutical formulation is used in a method for reducing the risk of cardiovascular death, myocardial infarction, stroke, and/or coronary revascularization in a mammalian subject with prior acute coronary syndrome.

In embodiments, the pharmaceutical formulation is used in a method for preventing a secondary cardiovascular event in a mammalian subject in need thereof. In embodiments, the pharmaceutical formulation is used in a method for reducing the risk of a major adverse cardiovascular event in a mammalian subject in need thereof. In embodiments, the pharmaceutical formulation is used in a method for increasing the concentration of high-density lipoprotein in the blood of a mammalian subject in need thereof.

In embodiments, the pharmaceutical formulation is used in a method for increasing one or more clinical endpoints related to high-density lipoprotein in a mammalian subject in need thereof. In this embodiment, the clinical endpoints can be one or more of HDL-c blood concentration, HDL particle number, HDL particle size, HDL phospholipid blood concentration, ApoA1 blood concentration and cholesterol efflux capacity. In embodiments, the pharmaceutical formulation is used in a method for decreasing inflammation in a mammalian subject in need thereof. In embodiments, the pharmaceutical formulation is used in a method for preventing inflammation in a mammalian subject in need thereof.

The methods embodied herein may be practiced using an injection device, including an autoinjection device as described above. In embodiments, the methods embodied herein may be practiced using a large volume bolus injector or a pre-filled syringe. In embodiments, the pre-filled syringe is an accessorized pre-filled syringe having one or more safety feature, e.g., a retracting needle after injection, and/or one or more ergonomics features, e.g., a finger flange or other accessory that assists in holding the syringe.

In embodiments, doses of the antibody that specifically binds to endothelial lipase for the methods embodied herein range from 50 mg to 400 mg per month. In embodiments, doses of the antibody that specifically binds to endothelial lipase for the methods embodied herein range from 30 mg to 500 mg per month. In embodiments, the dose of antibody is from 100 mg to 350 mg per month. In embodiments, the dose of antibody is from 250 mg to 350 mg per month. In embodiments, the dose of antibody is about 250 mg per month. In embodiments, the dose of antibody is 250 mg per month. In embodiments, the dose of antibody is about 125 mg per month. In embodiments, the dose of antibody is 125 mg per month. One skilled in the art will understand that the pharmaceutical formulation may need to be administered multiple times or at increased volumes to achieve the desired dose. For example, for a pharmaceutical formulation with an antibody concentration of 125 mg/mL may administered with 2 mL in a single injection or via two injections of 1 mL in order to achieve a dose of 250 mg. One skilled in the art will also understand that increasing the dosing frequency, such as increasing from monthly to bi-weekly, will allow for multiple injections to achieve a higher total dose.

In embodiments of the methods embodied herein, the mammalian subject is a human. In embodiments of the methods embodied herein, the mammalian subject is an animal such as an agricultural animal (e.g., cattle, sheep, swine), research animal (e.g., mice, rats, monkeys, chimpanzees) or companion animal (e.g., dogs, cats and rabbits).

### EXAMPLES

### Example 1: Characteristics of Anti-Endothelial Lipase Antibody MEDI5884

MEDI5884 is an IgG4P monoclonal antibody directed against endothelial lipase enzyme (SEQ ID NO: 13) involved in conversion of high density lipoprotein (HDL) to low density lipoprotein (LDL). The term “MEDI5884” refers to anti-EL antibody that comprises the heavy chain of SEQ ID NO:9 and the light chain of SEQ ID NO:10. MEDI5884 is also referred to as “S6F1-4P,” and it comprises the heavy chain variable region of the h55A1-S6 antibody and the light chain variable region of the h55A1-F1 antibody, which are disclosed in US Published Application No. 2017/0260290, which is herein incorporated by reference in its entirety. MEDI5884 has an overall molecular weight of approximately 150 kDa. The observed intact mass and peptide map are consistent with the expected primary structure, based on the DNA sequence. The heavy chain variable region of MEDI5884 is provided below as SEQ ID NO:4. The light chain variable region of MEDI5884 is provided below as SEQ ID NO:8. The full length heavy chain of MEDI5884 is provided below as SEQ ID NO:9. The full length light chain of MEDI5884 is provided below as SEQ ID NO:10. The experimentally determined extinction coefficient is 1.44 (mg/mL)⁻¹cm⁻¹ and the isoelectric point (pI) is in the range of 8.4-8.9. The first thermal transition as determined by differential scanning calorimetry is 56.4° C.

MEDI5884 binds to the endothelial lipase (EL) expressed on the surface of the Human Embryonic Kidney (HEK) 293 cells (the HEK 293 cell line has been engineered to express EL on the cell surface). The cell-bound MEDI5884 is then measured with a fixed concentration of an Eu-N1 anti-human IgG₄P, which binds to the Fc region of the MEDI5884. The Eu-N1 anti-human IgG₄P bound to MEDI5884 is measured by time-resolved fluorescence and the signal is directly proportional to MEDI5884 concentration.

### Example 2: Pre-Clinical Dose Formulation Development

MEDI5884 was evaluated through a pre-clinical developability study to understand the formulation development risks. The developability study was performed at 100 mg/ml in 20 mM His/His-HCl, 240 mM Sucrose, 0.02% (w/v) PS80, pH 6.0. Based on the developability study, MEDI5884 was found to be developable, however with a relatively high viscosity (12.6 cP at 23° C.) and K_{d} value (−19.6 mL/g). Based on this information and the anticipated clinical dose requirements (subcutaneous administration), a concentration of 100 mg/ml MEDI5884 following reconstitution of a lyophilized Drug Product (DP) was selected for use in a Phase 1 clinical trial.

The PS80 level in the drug substance (DS) was set to 0.02% (w/v), since this is known to provide adequate stability and protection from interfacial stress in most instances. However, the PS80 concentration in drug product (DP) post reconstitution is 0.04% (w/v) due to the half reconstitution approach.

### Formulation Development Approach

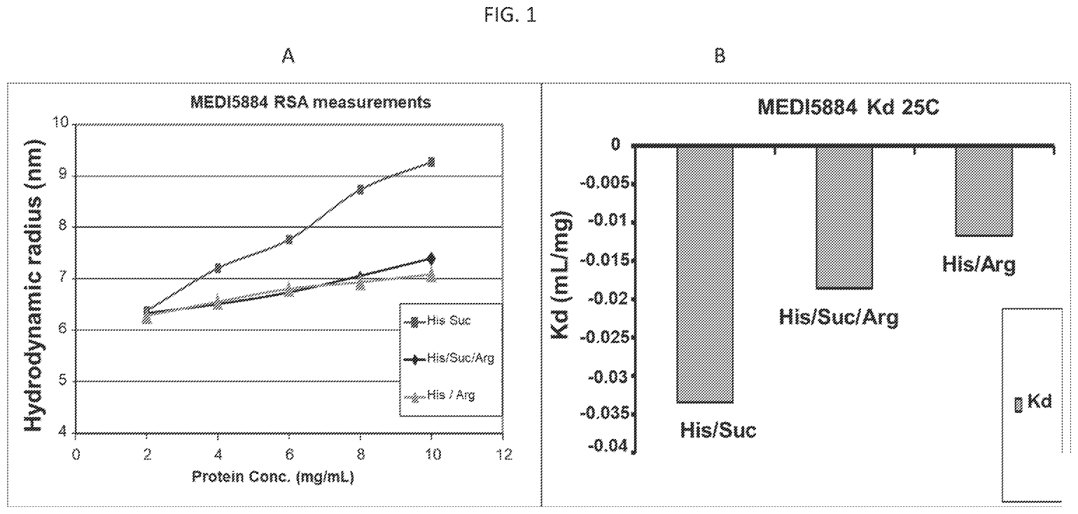

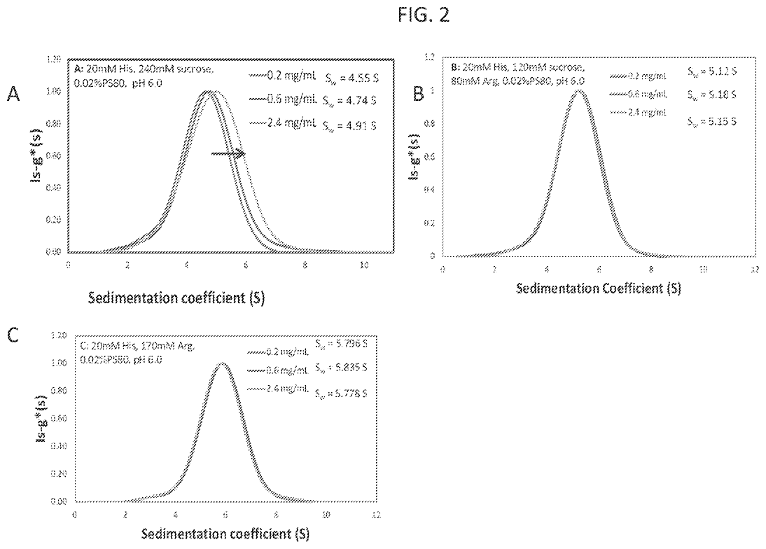

Initial formulation development was performed as summarized in Table 7 below. However, the drug substance (DS) formulation #1 showed precipitation and cloudiness during the tangential flow filtration (TFF) process. In addition, the drug product (DP) formulation #1 showed presence of reversible self-association (RSA) by Dynamic Light Scattering (DLS; FIG. 1) and Analytical Ultracentrifugation (AUC; FIG. 2). Therefore, a DS formulation #2 at 50 mg/mL was evaluated for Phase 1. Upon testing, the formulation #2 also showed cloudiness upon centrifugation. To mitigate the precipitation, cloudiness and RSA challenges, an Arginine formulation (#3) was then evaluated.

TABLE 7
Formulations evaluated for MEDI5884 Phase 1 Supply

| | DS/DP and | | |
|---|---|---|---|
| | Protein | | |
| Formulation | concentration | Formulation composition | Comment |
| #1 | DS at | 10 mM Histidine/Histidine- | Cloudiness and |
| | 50 mg/mL | HCl, 120 mM Sucrose, 0.02% | precipitation upon |
| | | (w/v) Polysorbate 80, pH 6.0 | TFF |
| | DP at | 20 mM Histidine/Histidine- | Presence of RSA |
| | 100 mg/mL | HCl, 240 mM Sucrose, 0.04% | |
| | | (w/v) Polysorbate 80, pH 6.0 | |
| #2 | DS at | 10 mM Histidine/Histidine- | Cloudiness upon |
| | 50 mg/mL | HCl, 60 mM Sucrose, 40 mM | centrifugation |
| | | Arginine-HCl, 0.02% (w/v) | |
| | | Polysorbate, pH 6.0 | |
| | DP at | 20 mM Histidine/Histidine- | No challenges |
| | 100 mg/mL | HCl, 120 mM Sucrose, 80 mM | for RSA |
| | | Arginine-HCl, 0.02% (w/v) | |
| | | Polysorbate, pH 6.0 | |
| #3 | DS at | 10 mM Histidine/Histidine- | No challenges |
| (Arginine | 50 mg/mL | HCl, 80 mM Arg-HCl, 0.02% | for TFF and |
| formulation) | | (w/v) Polysorbate 80, pH 6.0 | centrifugation |
| | | | process |
| | DP at | 20 mM Histidine/Histidine- | No challenges |
| | 100 mg/mL | HCl, 160 mM Arginine-HCl, | for RSA |
| | | 0.04% (w/v) Polysorbate 80, | |
| | | pH 6.0 | |

Based on results obtained with DLS & AUC, the Arginine DP formulation (#3) showed absence of RSA (FIG. 1A, FIG. 2C). In addition, the Arginine only DS formulation showed no challenges for TFF and the centrifugation process. Therefore, the Arginine formulation #3 (in both DS & DP form) of MEDI5884 was selected for further Phase 1 formulation development activities.

### SUMMARY

A lyophilized formulation of MEDI5884, at a composition (post reconstitution) of 100 mg/mL in 20 mM Histidine/Histidine-HCl, 160 mM Arginine-HCl, 0.04% (w/v) Polysorbate, pH 6.0, provides appropriate stability and solubility and was suitable as a formulation for phase 1 clinical trial material supply.

MEDI5884 can be stored long term as Drug Substance (DS; formulated with Polysorbate 80) and Unformulated Drug Substance (UDS; formulated without Polysorbate 80) in a Celsius pak at 2-8° C. and at −40° C., respectively.

### Example 3—Studies with Clinical Dose Formulation

### Materials

All the materials used for the study in this example were of USP or Multicompendial grade. All the solutions and buffers were prepared using USP or HPLC water and were filtered through 0.2 m PVDF filters (Millipore, Millex GV, SLG033RB) before further use. MEDI5884 samples for stability studies were prepared by following aseptic processing in a Biosafety Cabinet (BSC). Bulk material was stored at 2-8° C.

### Methods

### i) Protein Concentration Determination

MEDI5884 protein concentrations were determined by measuring absorbance at 280 nm with a Trinean HT-A280 spectrophotometer (Gentbrugge, Belgium). A measured extinction coefficient of 1.44 (mg/mL)−1 cm−1 was used to calculate protein concentrations. As measurement of MEDI5884 on the Trinean HT-A280 does not require dilution, density correction factors were not used.

### ii) Purity Determination by Size Exclusion Chromatography

Size Exclusion Chromatography (SEC) was used for purity determination by high pressure size exclusion chromatography (HPSEC). An Agilent HPLC system (Santa Clara, Calif.) with a TSK-Gel G3000 column was used for analysis. 250 μg of antibody was loaded on the column by injecting 25 ul of the antibody diluted to 10 mg/ml in PBS. Standard integration parameters were used for automatic integration of aggregate, monomer and fragment peaks.

iii) Visual Appearance

Visual inspection of the samples was performed by examining the samples in their respective container for particles, color, and clarity using appropriate standards.

### iv) Sub-Visible Particle Analysis

Sub-visible particles analysis was performed using light obscuration. For high accuracy (HIAC) analysis, samples were diluted to 5 mg/mL with formulation buffer and degassed for a minimum of 1 hour prior to testing.

### v) Osmolality

Osmolality was measured using Advanced Instrument Inc. 2020 freezing point depression osmometer (Norwood, Mass.). System suitability was assessed by running a reference standard.

### vi) Viscosity Assessment

The viscosity of MEDI5884 Drug Product at 100 mg/mL was measured using an Anton Paar MCR301 Rheometer (Graz, Austria) with 40 mm cone and plate accessory. Viscosities are reported at the high-shear limit of 1000 per second shear rate.

vii) Lyophilization of Product Vials

Lyophilization was performed using a Lyostar III lyophilizer (SP Scientific, Warminster, Pa.).

viii) Determination of the Glass Transition Temperature (Tg′) for Lyophilization

Tg′ was determined using a TA Instruments Differential Scanning Calorimetry Instrument, Model Q2000 (New Castle, Del.). About 20 μL of sample was loaded in the pan. The samples were cooled to −80° C. at 5° C./min. They were then heated to 20° C. at 5° C./min to pass through the glass transition temperature. The Tg′ was determined as the sigmoidal curve/inflection in the heat flow curve. Integration for Tg′ was performed using Universal Analysis software to determine the onset, mid and end of glass transition and midpoint Tg′ values were reported as (Tg′).

### ix) Moisture Determination of Lyophilized Product Using Karl Fischer Titration

The residual water present in the freeze-dried formulations was measured using Karl Fischer titration using a Mettler Toledo, model DL39 (Columbus, Ohio). Dry methanol was used to reconstitute the freeze-dried material. The residual water was determined by mass balance.

### x) Freeze-Drying Microscopy to Determine Collapse Temperature for Lyophilization

Freeze drying microscopy was performed using a liquid nitrogen vapor cooled Olympus Microscope (Tokyo, Japan) with sample holder capable of maintaining a vacuum on the sample. About 5-10 μL of sample was frozen to −40° C. and then the vacuum was started and the drying front of the cake was used to focus the microscope. Iterative ramp increases in temperature (fastest=2° C./min; slowest=0.5° C./min) were used to observe collapse in the microscope images of the drying cake. Onset of collapse (Tc) was reported as collapse temperature, which was observed as bright visible holes in the dried matrix at the sublimation front.

### xi) Formulation Stability Studies

Stability of MEDI5884 lyophilized Drug Product was assessed at 100 mg/ml formulated in 20 mM His/His-HCl, 160 mM Arg-HCl, 0.04% (w/v) PS80, pH 6.0. Samples were placed in stability chambers at 40° C./75% relative humidity (RH), 25° C./60% RH and 5° C.

### Data and Discussion

### a) Drug Substance and Unformulated Drug Substance Stability

i) DS and UDS Storage and Strategy

DS and UDS storage in Celsius pak at 2-8 and −40° C. respectively were selected to assess long-term storage stability of MEDI5884. Controlled Freeze/Thaw assessment was performed for DS and UDS in the Celsius-paks container.

i) Controlled DS & UDS Freeze Thaw (F/T) and Frozen Storage Assessment in Celsius Pak

Freeze thaw and frozen storage stability were assessed in a representative storage container (i.e., Celsius pak), temperature and process. Celsius paks were filled with UDS and DS and frozen and thawed in a controlled manner three times. After the completion of third thaw, the Celsius paks were frozen again and stored at −40 and 25° C. for long term stability assessment for DS and UDS. For UDS, long term stability was also performed at 2-8 C and one month frozen storage data is shown below in Table 8.

TABLE 8
DS Stability Assessment

| | | | | | | Sub-visible Particles | | |
|---|---|---|---|---|---|---|---|---|
| | | Visual | Purity (SEC) | | | (HIAC) | | |
| | Time | Inspection | Aggregate | Monomer | Fragment | ≥2 | ≥10 | ≥25 |
| DS | point | (Particles)* | % | % | % | μm | μm | μm |
| DS | T0 | <STD 1 | 0.9 | 99.1 | 0.0 | 4046 | 127 | 0 |
| | 3x F/T | <STD 1 | 0.9 | 99.1 | 0.0 | 24 | 1 | 0 |
| | Post3X FT, −40 | <STD 1 | 0.9 | 99.1 | 0.0 | 2227 | 87 | 0 |
| | C., 1 month | | | | | | | |
| | Post3X FT, 25 | <STD 1 | 0.9 | 99.1 | 0.0 | 31 | 1 | 0 |
| | C., 1 month | | | | | | | |

*Performed in vials;
<STD 1: practically free from visible particles;
<STD 5, 6, 7: visible particles present

DS showed no significant change in purity by size exclusion chromatography (SEC). Based on this assessment, tuED5884 is stable as a frozen DS in Celsius paks at −40° C. Also, there was no impact on stability for DS post 3×FT after 3 month of storage at 25° C.

Liquid Drug Substance stability is assessed in vials to support liquid hold times during storage, shipment and manufacturing. The liquid formulation stability was performed at 2-8, 25 and 40° C. in 3 cc glass vials with 1.0 mL fill volume. Results are shown in Table 9.

TABLE 9
Liquid Formulation Stability by HPSEC and Bioassay

| | 5° C. (based | 40° C. (based |
|---|---|---|
| Attribute | on 3 months) | on 3 months) |
| Monomer loss (%/month) | 0 | 1.2 |
| Aggregation (%/month) | 0 | 0.8 |
| Fragmentation (%/month) | 0 | 0.4 |

| | |
|---|---|
| Bioassay (after 6 month at 25 C.) | 108 |

There was no significant change in pH, osmolality or protein concentration during the study, and visible and sub-visible particles remained low.

### b) Viscosity Assessment

Viscosity measured at 23° C. was 5.4 cP for MEDI5884 at 100 mg/mL in 20 mM Histidine/Histidine-HCl, 160 mM Arginine-HCl, 0.04% (w/v) Polysorabte 80, pH 6.0. No challenges are expected in manufacturing or subcutaneous dose administration based on these viscosity measurements.

### C) Drug Product Stability Data

Drug product stability was assessed by SEC, visual inspection and melt flow index (MFI). Key results are reported below in Tables 10 and 11. These results support the use of this lyophilized drug product for Phase 1 clinical trials.

TABLE 10
Lyophilized Formulation Stability - SEC, and Visual Inspection

| | 5° C. (based | 40° C. (based |
|---|---|---|
| Attribute | on 5 months) | on 3 months) |
| Monomer loss (%/month) | 0.06 | 1.7 |
| Aggregation (%/month) | 0.06 | 1.7 |
| Fragmentation (%/month) | 0 | 0 |
| Max particles observed by VI | <STD 1 | <STD 1 |

TABLE 11
Lyophilized Formulation Stability- Sub-visible Particles by MFI

| | Particles per container | | |
|---|---|---|---|
| | ≥2 μm | ≥10 μm | ≥25 μm |
| T0 | 118311 | 615 | 46 |
| 1 months 25° C. | 122090 | 306 | 50 |
| 1 months 5° C. | 114156 | 88 | 4 |
| 3 months 5° C. | 137862 | 615 | 8 |

pH, osmolality and protein concentration remained unchanged during the DP stability study. Reconstitution time was in the range of 4 to 10 mins across the stability study and did not change significantly with time on stability. Moisture content for lyophilized cake was <1% for all samples in the study.

### d) Post-Reconstitution Stability

Post-reconstitution stability was assessed by SEC, visual inspection and MFI in order to support in-use stability in the clinic as well as hold times for analytical testing. Vials were reconstituted and tested at t0 and material was subsequently held in the vial at 5° C. and 25° C. for up to 1 week. Stability is maintained in both cases with no significant change in monomer purity and low visible and sub-visible particle levels comparable to t0. Data are shown in Table 12 below. Protein concentration, pH and osmolality were unchanged during the study (data not shown). Therefore, the material can be held at 5° C. up to 7 days without concerns regarding stability of reconstituted drug product.

TABLE 12
Post-reconstitution Stability

| | | | | | Subvisible Particles | | |
|---|---|---|---|---|---|---|---|
| | Visual | Purity by SEC | | | per mL by MFI | | |
| Time | Inspection | % | % | % | ≥2 | ≥10 | ≥25 |
| point/Temperature | (particles) | Aggregate | monomer | Fragment | μm | μm | μm |
| T0 | <STD 1 | 1.1 | 98.9 | 0 | 275 | 8 | 0 |
| 5° C. 7 days | <STD 1 | 1.1 | 98.9 | 0 | 1895 | 8 | 4 |
| 25° C. 7 days | <STD 1 | 1.1 | 98.9 | 0 | 1694 | 0 | 0 |

### Example 4—Development of Formulations of Medi5884 Suitable for Use in Autoinjector Devices

This example describes the development of formulations of MEDI5884 having suitable viscosities for use in autoinjector devices for subcutaneous administration. Certain parameters of the formulation were tested for their effect on the viscosity of the formulation. All methods performed in this example were performed as described in Example 3 unless otherwise indicated.

a) pH Buffer Screen

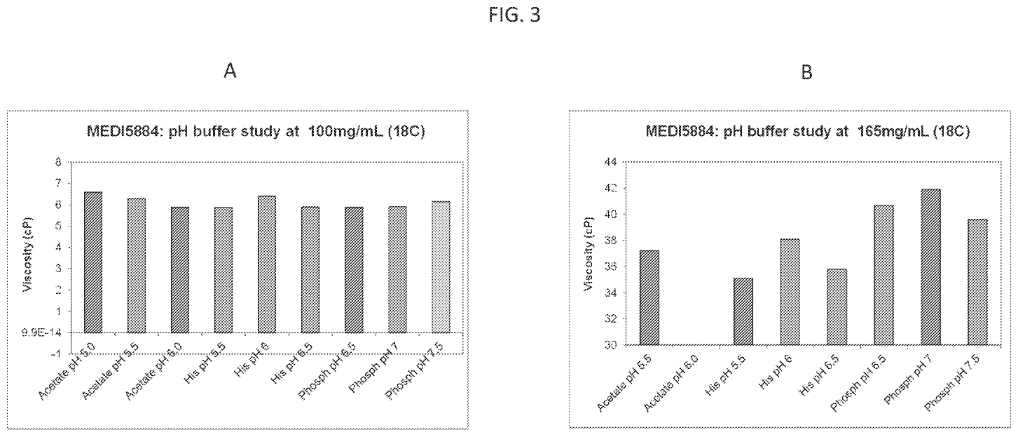

This study was performed in order to determine a suitable pH for the product formulation. Formulations were tested for the viscosity effects of three different buffers each at three different pH values. Formulations tested contained either 100 mg/mL or 165 mg/mL MEDI5884 in 160 mM ArgHCl and 20 mm of the buffer indicated at the pH indicated. Tests were performed at 18° C. Results are shown in FIG. 3, with FIG. 3A showing results for the 100 mg/mL MEDI5884 formulation and FIG. 3B showing results for the 165 mg/mL MEDI5884 formulation.

b) Excipient Screen

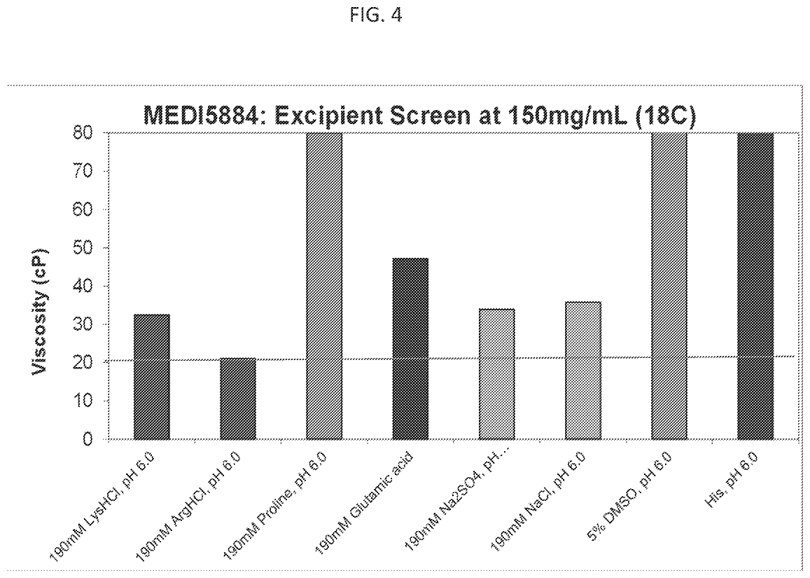

Formulations were tested for the viscosity effects of various excipients. The formulations tested had a MEDI5884 concentration of 150 mg/mL in 20 mM His/His-HCl at pH 6.0. Tests were performed at 18° C. All excipients were present at a concentration of 190 mM except for DMSO, which was at a concentration of 5%. A Histidine only formulation was tested having no additional excipient added. Excipients tested were: the positively charged amino acids lysine HCl (Lys HCl) and arginine HCl (Arg HCl); the hydrophobic amino acid proline; the negatively charged amino acid glutamic acid (as sodium glutamate); the salts sodium sulfate (Na₂SO₄) and sodium chloride (NaCl); and the cosolvent dimethyl sulfoxide (DMSO). Results of the excipient screen are shown in FIG. 4. As can be seen in FIG. 4, ArgHCl showed a significant decrease in viscosity at 150 mg/mL protein concentration compared to other excipients.

C) Determination of Viscosity at Different ArgHCl Concentrations

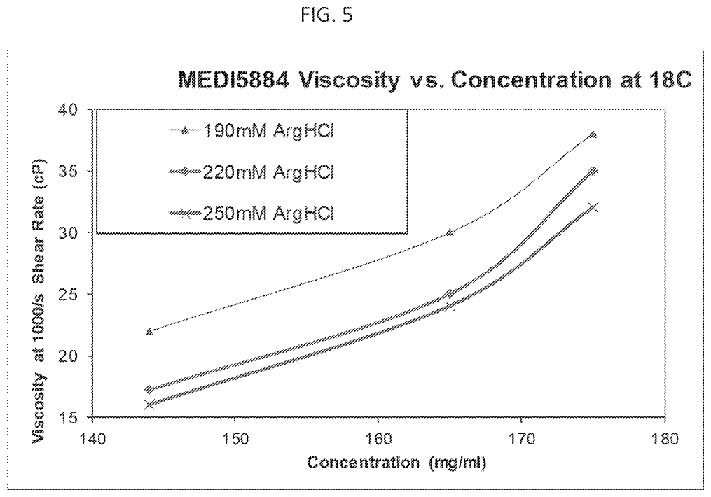

Formulations were tested for the viscosity effects of different concentrations of ArgHCl excipient in order to determine a suitable concentration of ArgHCl. The formulations tested had a MEDI5884 concentration of 145, 165 or 175 mg/mL in 20 mM His/HisHCl 0.02% PS80 and the concentrations of ArgHCl indicated. Formulations had a pH of 6.0 and were tested at 18° C. Results of this viscosity screening are shown in FIG. 5. As can been seen in FIG. 5, no significant decrease in viscosity for MEDI5884 is achieved by increasing the Arg HCl concentration from 220 mM to 250 mM.

d) pH Robustness Screen

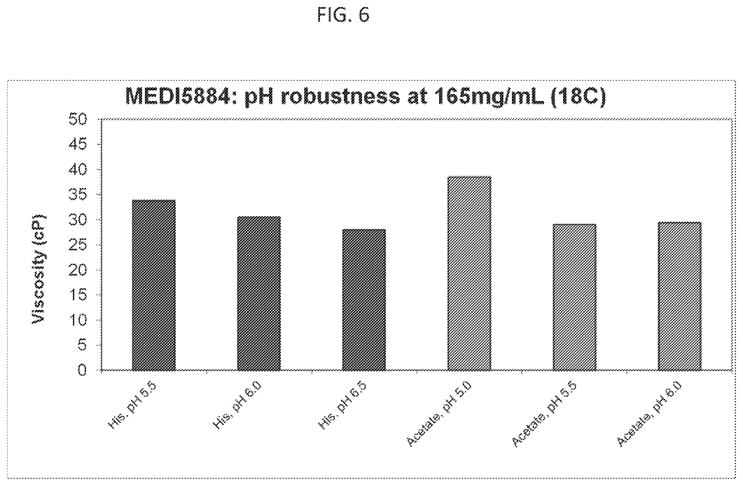

Formulations were tested for the effects of variations in pH. The formulations tested had a MEDI5884 concentration of 165 mg/mL in 20 mM His/HisHCl and 220 mM ArgHCl in either His or acetate buffer at the pH values indicated. Tests were performed at 18° C. Results of the pH robustness screen are shown in FIG. 6. As can be seen from FIG. 6, no major impact of pH 6.0±0.5 on viscosity was seen at a MEDI5884 concentration of 165 mg/mL.

e) Determination of Viscosity at Different MEDI5884 Concentrations

Formulations having concentrations of greater than 100 mg/mL MEDI5884 may be needed for use in autoinjector delivery systems. This viscosity study was conducted in order to determine suitable concentrations of MEDI5884 in the formulation. The viscosity and glide force (GF) of formulations having varying concentrations of MEDI5884 were tested at concentrations of 135, 150 and 165 mg/mL MEDI5884 in 20 mM His/His-HCl, 220 mM Arg-HCl, 0.02% (w/v) PS80 at pH 6.0. Viscosity at 18° C. was measured using the methods described in Example 3. Glide force was also measured using pre-filled syringes (PFS) containing the formulation at 18° C. using Instron. Results are shown in Table 13.

TABLE 13
viscosity and glide force results

| Protein | Viscosity (cP) at 18° C. | Avg. GF (N) at 18° C. | |
|---|---|---|---|
| conc.(mg/mL) | (at 1000 s⁻¹) | 1 mL PFS | 2 mL PFS |
| 135 | 12.8 | 9.3 | 18.8 |
| 150 | 21.8 | 11.9 | 28.1 |
| 165 | 36.8 | 16.4 | 42.8 |

Current Avg. GF limit for 1 mL pre-filled syringe (PFS) = 20N ; GF = Glide force; n = 10 for Avg. GF measurement.

f) Drug Product Stability Studies

The stability of drug product (DP) formulations was tested using the stability study methods described in Example 3. As the target formulation had a MED5884 concentration of 125 mg/mL, bracketing concentrations of 110 mg/mL and 140 mg/mL were used. Each formulation had a concentration of 20 mM Histidine/Histidine HCl, 220 mM Arg-HCl and 0.02% (w/v) PS80 at pH 6.0. Studies were performed at 5° C. for 16 months, 25° C. for 6 months and 40° C. for 3 months. The rate of monomer loss per month, percent aggregation per month and fragmentation per month were all measured. Results are shown in Table 14 below.

TABLE 14
Summary of stability studies

| Formulation | | Rate of | % | % |
|---|---|---|---|---|
| (Target | Stability | monomer loss | Aggregate/ | Fragment/ |
| 125 mg/mL) | Study | (%)/month | month | month |
| 140 mg/mL | 5° C., 16 M | 0.0 | 0.0 | 0.0 |
| MEDI5884 | 25° C., 6 M | 0.06 | 0.04 | 0.02 |
| | 40° C., 3 M | 1.8 | 1.6 | 0.2 |
| 110 mg/mL | 5° C., 16 M | 0.01 | 0.01 | 0.0 |
| MEDI5884 | 25° C., 6 M | 0.05 | 0.03 | 0.02 |
| | 40° C., 3 M | 1.6 | 1.4 | 0.2 |

As can be seen in Table 14, no significant change in monomer purity was seen after 16 months at 2-8 C, and there were low aggregation and fragmentation rates at 25° C. and 40° C.

It will be readily apparent to one of ordinary skill in the relevant arts that other suitable modifications and adaptations to the methods and applications described herein can be made without departing from the scope of any of the embodiments. The above examples are included herewith for purposes of illustration only and are not intended to be limiting.

It is to be understood that while certain embodiments have been illustrated and described herein, the claims are not to be limited to the specific forms or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may be practiced otherwise than as specifically described.

While various embodiments have been described above, it should be understood that they have been presented only as illustrations and examples of the present technology, and not by way of limitation. It will be apparent to persons skilled in the relevant art that various changes in form and detail can be made therein without departing from the spirit and scope of the present technology. Thus, the breadth and scope of the present technology should not be limited by any of the above-described embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

SEQUENCES

| SEQ | | |
|---|---|---|
| ID | Antibody | |
| NO | region | Sequence |
| 1 | HCDR1 | Asn Tyr Ala Leu Asn |
| 2 | HCDR2 | Trp Ile Asn Thr Tyr Ser Gly Val Gly Thr |
| | | Tyr Ala Gly Glu Phe Lys Gly |
| 3 | HCDR3 | Arg Gly Phe Tyr Gly Arg Arg Phe Phe Asp |
| | | Val |
| 4 | VH | Gln Val Gln Leu Val Gln Ser Gly Ser Glu |
| | | Leu Lys Lys Pro Gly Ala Ser Val Lys Val |
| | | Ser Cys Lys Ala Ser Gly Tyr Thr Phe Thr |
| | | Asn Tyr Ala Leu Asn Trp Val Arg Gln Ala |
| | | Pro Gly Gln Gly Leu Glu Trp Met Gly Trp |
| | | Ile Asn Thr Tyr Ser Gly Val Gly Thr Tyr |
| | | Ala Gly Glu Phe Lys Gly Arg Phe Val Phe |
| | | Ser Leu Asp Thr Ser Val Ser Thr Ala Tyr |
| | | Leu Gln Ile Ser Ser Leu Lys Ala Glu Asp |
| | | Thr Ala Val Tyr Tyr Cys Ala Arg Arg Gly |
| | | Phe Tyr Gly Arg Arg Phe Phe Asp Val Trp |
| | | Gly Lys Gly Thr Thr Val Thr Val Ser Ser |
| 5 | LCDR1 | Lys Ala Ser Gln Ser Val Asp Tyr Asp Val |
| | | Asp Ser Tyr Met His |
| 6 | LCDR2 | Ala Ala Ser Asn Leu Ala Ser |
| 7 | LCDR3 | Gln Gln Thr Ile Glu Asp Pro Pro Thr |
| 8 | VL | Asp Ile Gln Leu Thr Gln Ser Pro Ser Ser |
| | | Leu Ser Ala Ser Val Gly Asp Arg Val Thr |
| | | Ile Thr Cys Lys Ala Ser Gln Ser Val Asp |
| | | Tyr Asp Val Asp Ser Tyr Met His Trp Tyr |
| | | Gln Gln Lys Pro Gly Lys Ala Pro Lys Leu |
| | | Leu Ile Tyr Ala Ala Ser Asn Leu Ala Ser |
| | | Gly Val Pro Ser Arg Phe Ser Gly Ser Gly |
| | | Ser Gly Thr Asp Phe Thr Phe Thr Ile Ser |
| | | Ser Leu Gln Pro Glu Asp Ile Ala Thr Tyr |
| | | Tyr Cys Gln Gln Thr Ile Glu Asp Pro Pro |
| | | Thr Phe Gly Gly Gly Thr Lys Val Glu Ile |
| | | Lys |
| 9 | Full length | Gln Val Gln Leu Val Gln Ser Gly Ser Glu |
| | heavy | Leu Lys Lys Pro Gly Ala Ser Val Lys Val |
| | chain | Ser Cys Lys Ala Ser Gly Tyr Thr Phe Thr |
| | | Asn Tyr Ala Leu Asn Trp Val Arg Gln Ala |
| | | Pro Gly Gln Gly Leu Glu Trp Met Gly Trp |
| | | Ile Asn Thr Tyr Ser Gly Val Gly Thr Tyr |
| | | Ala Gly Glu Phe Lys Gly Arg Phe Val Phe |
| | | Ser Leu Asp Thr Ser Val Ser Thr Ala Tyr |
| | | Leu Gln Ile Ser Ser Leu Lys Ala Glu Asp |
| | | Thr Ala Val Tyr Tyr Cys Ala Arg Arg Gly |
| | | Phe Tyr Gly Arg Arg Phe Phe Asp Val Trp |
| | | Gly Lys Gly Thr Thr Val Thr Val Ser Ser |
| | | Ala Ser Thr Lys Gly Pro Ser Val Phe Pro |
| | | Leu Ala Pro Cys Ser Arg Ser Thr Ser Glu |
| | | Ser Thr Ala Ala Leu Gly Cys Leu Val Lys |
| | | Asp Tyr Phe Pro Glu Pro Val Thr Val Ser |
| | | Trp Asn Ser Gly Ala Leu Thr Ser Gly Val |
| | | His Thr Phe Pro Ala Val Leu Gln Ser Ser |
| | | Gly Leu Tyr Ser Leu Ser Ser Val Val Thr |
| | | Val Pro Ser Ser Ser Leu Gly Thr Lys Thr |
| | | Tyr Thr Cys Asn Val Asp His Lys Pro Ser |
| | | Asn Thr Lys Val Asp Lys Arg Val Glu Ser |
| | | Lys Tyr Gly Pro Pro Cys Pro Pro Cys Pro |
| | | Ala Pro Glu Phe Leu Gly Gly Pro Ser Val |
| | | Phe Leu Phe Pro Pro Lys Pro Lys Asp Thr |
| | | Leu Met Ile Ser Arg Thr Pro Glu Val Thr |
| | | Cys Val Val Val Asp Val Ser Gln Glu Asp |
| | | Pro Glu Val Gln Phe Asn Trp Tyr Val Asp |
| | | Gly Val Glu Val His Asn Ala Lys Thr Lys |
| | | Pro Arg Glu Glu Gln Phe Asn Ser Thr Tyr |
| | | Arg Val Val Ser Val Leu Thr Val Leu His |
| | | Gln Asp Trp Leu Asn Gly Lys Glu Tyr Lys |
| | | Cys Lys Val Ser Asn Lys Gly Leu Pro Ser |
| | | Ser Ile Glu Lys Thr Ile Ser Lys Ala Lys |
| | | Gly Gln Pro Arg Glu Pro Gln Val Tyr Thr |
| | | Leu Pro Pro Ser Gln Glu Glu Met Thr Lys |
| | | Asn Gln Val Ser Leu Thr Cys Leu Val Lys |
| | | Gly Phe Tyr Pro Ser Asp Ile Ala Val Glu |
| | | Trp Glu Ser Asn Gly Gln Pro Glu Asn Asn |
| | | Tyr Lys Thr Thr Pro Pro Val Leu Asp Ser |
| | | Asp Gly Ser Phe Phe Leu Tyr Ser Arg Leu |
| | | Thr Val Asp Lys Ser Arg Trp Gln Glu Gly |
| | | Asn Val Phe Ser Cys Ser Val Met His Glu |
| | | Ala Leu His Asn His Tyr Thr Gln Lys Ser |
| | | Leu Ser Leu Ser Leu Gly Lys |
| 10 | Full length | Asp Ile Gln Leu Thr Gln Ser Pro Ser Ser |
| | light chain | Leu Ser Ala Ser Val Gly Asp Arg Val Thr |
| | | Ile Thr Cys Lys Ala Ser Gln Ser Val Asp |
| | | Tyr Asp Val Asp Ser Tyr Met His Trp Tyr |
| | | Gln Gln Lys Pro Gly Lys Ala Pro Lys Leu |
| | | Leu Ile Tyr Ala Ala Ser Asn Leu Ala Ser |
| | | Gly Val Pro Ser Arg Phe Ser Gly Ser Gly |
| | | Ser Gly Thr Asp Phe Thr Phe Thr Ile Ser |
| | | Ser Leu Gln Pro Glu Asp Ile Ala Thr Tyr |
| | | Tyr Cys Gln Gln Thr Ile Glu Asp Pro Pro |
| | | Thr Phe Gly Gly Gly Thr Lys Val Glu Ile |
| | | Lys Arg Thr Val Ala Ala Pro Ser Val Phe |
| | | Ile Phe Pro Pro Ser Asp Glu Gln Leu Lys |
| | | Ser Gly Thr Ala Ser Val Val Cys Leu Leu |
| | | Asn Asn Phe Tyr Pro Arg Glu Ala Lys Val |
| | | Gln Trp Lys Val Asp Asn Ala Leu Gln Ser |
| | | Gly Asn Ser Gln Glu Ser Val Thr Glu Gln |
| | | Asp Ser Lys Asp Ser Thr Tyr Ser Leu Ser |
| | | Ser Thr Leu Thr Leu Ser Lys Ala Asp Tyr |
| | | Glu Lys His Lys Val Tyr Ala Cys Glu Val |
| | | Thr His Gln Gly Leu Ser Ser Pro Val Thr |
| | | Lys Ser Phe Asn Arg Gly Glu Cys |
| 11 | Human | Ala Ser Thr Lys Gly Pro Ser Val Phe Pro |
| | IgG4P | Leu Ala Pro Cys Ser Arg Ser Thr Ser Glu |
| | heavy | Ser Thr Ala Ala Leu Gly Cys Leu Val Lys |
| | chain | Asp Tyr Phe Pro Glu Pro Val Thr Val Ser |
| | constant | Trp Asn Ser Gly Ala Leu Thr Ser Gly Val |
| | region | His Thr Phe Pro Ala Val Leu Gln Ser Ser |
| | sequence | Gly Leu Tyr Ser Leu Ser Ser Val Val Thr |
| | | Val Pro Ser Ser Ser Leu Gly Thr Lys Thr |
| | | Tyr Thr Cys Asn Val Asp His Lys Pro Ser |
| | | Asn Thr Lys Val Asp Lys Arg Val Glu Ser |
| | | Lys Tyr Gly Pro Pro Cys Pro Pro Cys Pro |
| | | Ala Pro Glu Phe Leu Gly Gly Pro Ser Val |
| | | Phe Leu Phe Pro Pro Lys Pro Lys Asp Thr |
| | | Leu Met Ile Ser Arg Thr Pro Glu Val Thr |
| | | Cys Val Val Val Asp Val Ser Gln Glu Asp |
| | | Pro Glu Val Gln Phe Asn Trp Tyr Val Asp |
| | | Gly Val Glu Val His Asn Ala Lys Thr Lys |
| | | Pro Arg Glu Glu Gln Phe Asn Ser Thr Tyr |
| | | Arg Val Val Ser Val Leu Thr Val Leu His |
| | | Gln Asp Trp Leu Asn Gly Lys Glu Tyr Lys |
| | | Cys Lys Val Ser Asn Lys Gly Leu Pro Ser |
| | | Ser Ile Glu Lys Thr Ile Ser Lys Ala Lys |
| | | Gly Gln Pro Arg Glu Pro Gln Val Tyr Thr |
| | | Leu Pro Pro Ser Gln Glu Glu Met Thr Lys |
| | | Asn Gln Val Ser Leu Thr Cys Leu Val Lys |
| | | Gly Phe Tyr Pro Ser Asp Ile Ala Val Glu |
| | | Trp Glu Ser Asn Gly Gln Pro Glu Asn Asn |
| | | Tyr Lys Thr Thr Pro Pro Val Leu Asp Ser |
| | | Asp Gly Ser Phe Phe Leu Tyr Ser Arg Leu |
| | | Thr Val Asp Lys Ser Arg Trp Gln Glu Gly |
| | | Asn Val Phe Ser Cys Ser Val Met His Glu |
| | | Ala Leu His Asn His Tyr Thr Gln Lys Ser |
| | | Leu Ser Leu Ser Leu Gly Lys |
| 12 | Human C | Arg Thr Val Ala Ala Pro Ser Val Phe Ile |
| | kappa light | Phe Pro Pro Ser Asp Glu Gln Leu Lys Ser |
| | chain | Gly Thr Ala Ser Val Val Cys Leu Leu Asn |
| | constant | Asn Phe Tyr Pro Arg Glu Ala Lys Val Gln |
| | region | Trp Lys Val Asp Asn Ala Leu Gln Ser Gly |
| | sequence | Asn Ser Gln Glu Ser Val Thr Glu Gln Asp |
| | | Ser Lys Asp Ser Thr Tyr Ser Leu Ser Ser |
| | | Thr Leu Thr Leu Ser Lys Ala Asp Tyr Glu |
| | | Lys His Lys Val Tyr Ala Cys Glu Val Thr |
| | | His Gln Gly Leu Ser Ser Pro Val Thr Lys |
| | | Ser Phe Asn Arg Gly Glu Cys |
| 13 | Mature | Ser Pro Val Pro Phe Gly Pro Glu Gly Arg |
| | Human | Leu Glu Asp Lys Leu His Lys Pro Lys Ala |
| | Endothelial | Thr Gln Thr Glu Val Lys Pro Ser Val Arg |
| | Lipase | Phe Asn Leu Arg Thr Ser Lys Asp Pro Glu |
| | (lacking | His Glu Gly Cys Tyr Leu Ser Val Gly His |
| | leader | Ser Gln Pro Leu Glu Asp Cys Ser Phe Asn |
| | sequence): | Met Thr Ala Lys Thr Phe Phe Ile Ile His |
| | | Gly Trp Thr Met Ser Gly Ile Phe Glu Asn |
| | | Trp Leu His Lys Leu Val Ser Ala Leu His |
| | | Thr Arg Glu Lys Asp Ala Asn Val Val Val |
| | | Val Asp Trp Leu Pro Leu Ala His Gln Leu |
| | | Tyr Thr Asp Ala Val Asn Asn Thr Arg Val |
| | | Val Gly His Ser Ile Ala Arg Met Leu Asp |
| | | Trp Leu Gln Glu Lys Asp Asp Phe Ser Leu |
| | | Gly Asn Val His Leu Ile Gly Tyr Ser Leu |
| | | Gly Ala His Val Ala Gly Tyr Ala Gly Asn |
| | | Phe Val Lys Gly Thr Val Gly Arg Ile Thr |
| | | Gly Leu Asp Pro Ala Gly Pro Met Phe Glu |
| | | Gly Ala Asp Ile His Lys Arg Leu Ser Pro |
| | | Asp Asp Ala Asp Phe Val Asp Val Leu His |
| | | Thr Tyr Thr Arg Ser Phe Gly Leu Ser Ile |
| | | Gly Ile Gln Met Pro Val Gly His Ile Asp |
| | | Ile Tyr Pro Asn Gly Gly Asp Phe Gln Pro |
| | | Gly Cys Gly Leu Asn Asp Val Leu Gly Ser |
| | | Ile Ala Tyr Gly Thr Ile Thr Glu Val Val |
| | | Lys Cys Glu His Glu Arg Ala Val His Leu |
| | | Phe Val Asp Ser Leu Val Asn Gln Asp Lys |
| | | Pro Ser Phe Ala Phe Gln Cys Thr Asp Ser |
| | | Asn Arg Phe Lys Lys Gly Ile Cys Leu Ser |
| | | Cys Arg Lys Asn Arg Cys Asn Ser Ile Gly |
| | | Tyr Asn Ala Lys Lys Met Arg Asn Lys Arg |
| | | Asn Ser Lys Met Tyr Leu Lys Thr Arg Ala |
| | | Gly Met Pro Phe Arg Val Tyr His Tyr Gln |
| | | Met Lys Ile His Val Phe Ser Tyr Lys Asn |
| | | Met Gly Glu Ile Glu Pro Thr Phe Tyr Val |
| | | Thr Leu Tyr Gly Thr Asn Ala Asp Ser Gln |
| | | Thr Leu Pro Leu Glu Ile Val Glu Arg Ile |
| | | Glu Gln Asn Ala Thr Asn Thr Phe Leu Val |
| | | Tyr Thr Glu Glu Asp Leu Gly Asp Leu Leu |
| | | Lys Ile Gln Leu Thr Trp Glu Gly Ala Ser |
| | | Gln Ser Trp Tyr Asn Leu Trp Lys Glu Phe |
| | | Arg Ser Tyr Leu Ser Gln Pro Arg Asn Pro |
| | | Gly Arg Glu Leu Asn Ile Arg Arg Ile Arg |
| | | Val Lys Ser Gly Glu Thr Gln Arg Lys Leu |
| | | Thr Phe Cys Thr Glu Asp Pro Glu Asn Thr |
| | | Ser Ile Ser Pro Gly Arg Glu Leu Trp Phe |
| | | Arg Lys Cys Arg Asp Gly Trp Arg Met Lys |
| | | Asn Glu Thr Ser Pro Thr Val Glu Leu Pro |

HCDR = Heavy chain complementarity determining region
VH = Heavy chain variable domain
LCDR = Light chain complementarity determining region
VL = Light chain variable domain

What is claimed is:

1. A pharmaceutical formulation comprising:
(i) about 40 mg/mL to about 160 mg/mL of an antibody that specifically binds to human endothelial lipase, and
(ii) about 50 mM to about 260 mM arginine salt.

2. The pharmaceutical formulation of claim 1, comprising about 100 mg/mL to about 150 mg/mL of the antibody that specifically binds to endothelial lipase.

3. The pharmaceutical formulation of claim 1, comprising about 110 mg/mL to about 140 mg/mL of the antibody that specifically binds to endothelial lipase.

4. The pharmaceutical formulation of claim 1, comprising about 120 mg/mL to about 130 mg/mL of the antibody that specifically binds to endothelial lipase.

5. The pharmaceutical formulation of claim 1, comprising about 125 mg/mL of the antibody that specifically binds to endothelial lipase.

6. The pharmaceutical formulation of claim 1, comprising about 40 mg/mL to about 60 mg/mL of the antibody that specifically binds to endothelial lipase.

7. The pharmaceutical formulation of claim 1, comprising about 50 mg/mL of the antibody that specifically binds to endothelial lipase.

8. The pharmaceutical formulation of claim 1, comprising about 90 mg/mL to about 110 mg/mL of the antibody that specifically binds to endothelial lipase.

9. The pharmaceutical formulation of claim 1, comprising about 100 mg/mL of the antibody that specifically binds to endothelial lipase.

10. The pharmaceutical formulation of any one of claims 1 to 9, wherein the arginine salt comprises arginine hydrochloride, arginine acetate, arginine glutamate, or arginine sulfate.

11. The pharmaceutical formulation of any one of claims 1 to 9, wherein the arginine salt is arginine hydrochloride.

12. The pharmaceutical formulation of any one of claims 1 to 11, comprising about 160 mM to about 240 mM arginine hydrochloride.

13. The pharmaceutical formulation of any one of claims 1 to 11, comprising about 180 mM to about 230 mM arginine hydrochloride.

14. The pharmaceutical formulation of any one of claims 1 to 11, comprising about 200 mM to about 225 mM arginine hydrochloride.

15. The pharmaceutical formulation of any one of claims 1 to 11 comprising about 220 mM arginine hydrochloride.

16. The pharmaceutical formulation of any one of claims 1 to 11, comprising about 110 mM to about 140 mM arginine hydrochloride.

17. The pharmaceutical formulation of any one of claims 1 to 11, comprising about 50 mM to about 110 mM arginine hydrochloride.

18. The pharmaceutical formulation of any one of claims 1 to 11, comprising about 60 mM to about 100 mM arginine hydrochloride.

19. The pharmaceutical formulation of any one of claims 1 to 11, comprising about 70 mM to about 90 mM arginine hydrochloride.

20. The pharmaceutical formulation of any one of claims 1 to 11, comprising about 80 mM arginine hydrochloride.

21. The pharmaceutical formulation of any one of claims 1 to 20, wherein the antibody that specifically binds endothelial lipase has a first heavy chain complementarity determining region (CDR1) with an amino acid sequence at least 80% identical to SEQ ID NO: 1.

22. The pharmaceutical formulation of any one of claims 1 to 21, wherein the antibody that specifically binds endothelial lipase has a heavy chain CDR1 with an amino acid sequence comprising SEQ ID NO: 1.

23. The pharmaceutical formulation of any one of claims 1 to 22, wherein the antibody that specifically binds endothelial lipase has a second heavy chain complementarity determining region (CDR2) with an amino acid sequence at least 90% identical to SEQ ID NO: 2.

24. The pharmaceutical formulation of any one of claims 1 to 23, wherein the antibody that specifically binds endothelial lipase has a heavy chain CDR2 with an amino acid sequence comprising SEQ ID NO: 2.

25. The pharmaceutical formulation of any one of claims 1 to 24, wherein the antibody that specifically binds endothelial lipase has a third heavy chain complementarity determining region (CDR3) with an amino acid sequence at least 90% identical to SEQ ID NO: 3.

26. The pharmaceutical formulation of any one of claims 1 to 25, wherein the antibody that specifically binds endothelial lipase has a heavy chain CDR3 with an amino acid sequence comprising SEQ ID NO: 3.

27. The pharmaceutical formulation of any one of claims 1 to 26, wherein the antibody that specifically binds endothelial lipase has a first light chain complementarity determining region (CDR1) with an amino acid sequence at least 90% identical to SEQ ID NO: 5.

28. The pharmaceutical formulation of any one of claims 1 to 27, wherein the antibody that specifically binds endothelial lipase has a light chain CDR1 with an amino acid sequence comprising SEQ ID NO: 5.

29. The pharmaceutical formulation of any one of claims 1 to 28, wherein the antibody that specifically binds endothelial lipase has a second light chain complementarity determining region (CDR2) with an amino acid sequence at least 80% identical to SEQ ID NO: 6.

30. The pharmaceutical formulation of any one of claims 1 to 29, wherein the antibody that specifically binds endothelial lipase has a light chain CDR2 with an amino acid sequence comprising SEQ ID NO: 6.

31. The pharmaceutical formulation of any one of claims 1 to 30, wherein the antibody that specifically binds endothelial lipase has a third light chain complementarity determining region (CDR3) with an amino acid sequence at least 80% identical to SEQ ID NO: 7.

32. The pharmaceutical formulation of any one of claims 1 to 31, wherein the antibody that specifically binds endothelial lipase has a light chain CDR3 with an amino acid sequence comprising SEQ ID NO: 7.

33. The pharmaceutical formulation of any one of claims 1 to 20, wherein the heavy chain of the antibody that specifically binds to endothelial lipase comprises an amino acid sequence at least 90% identical to SEQ ID NO: 4.

34. The pharmaceutical formulation of any one of claims 1 to 20, wherein the heavy chain of the antibody that specifically binds to endothelial lipase comprises an amino acid sequence at least 95% identical to SEQ ID NO: 4.

35. The pharmaceutical formulation of any one of claims 1 to 20, wherein the heavy chain of the antibody that specifically binds to endothelial lipase comprises an amino acid sequence at least 98% identical to SEQ ID NO: 4.

36. The pharmaceutical formulation of any one of claims 1 to 20, wherein the heavy chain of the antibody that specifically binds to endothelial lipase comprises an amino acid sequence at least 99% identical to SEQ ID NO: 4.

37. The pharmaceutical formulation of any one of claims 1 to 20, wherein the heavy chain of the antibody that specifically binds to endothelial lipase comprises SEQ ID NO: 4.

38. The pharmaceutical formulation of any one of claims 1 to 37, wherein the light chain of the antibody that specifically binds to endothelial lipase comprises an amino acid sequence at least 90% identical to SEQ ID NO: 8.

39. The pharmaceutical formulation of any one of claims 1 to 37, wherein the light chain of the antibody that specifically binds to endothelial lipase comprises an amino acid sequence at least 95% identical to SEQ ID NO: 8.

40. The pharmaceutical formulation of any one of claims 1 to 37, wherein the light chain of the antibody that specifically binds to endothelial lipase comprises an amino acid sequence at least 98% identical to SEQ ID NO: 8.

41. The pharmaceutical formulation of any one of claims 1 to 37, wherein the light chain of the antibody that specifically binds to endothelial lipase comprises an amino acid sequence at least 99% identical to SEQ ID NO: 8.

42. The pharmaceutical formulation of any one of claims 1 to 37, wherein the light chain of the antibody that specifically binds to endothelial lipase comprises SEQ ID NO: 8.

43. The pharmaceutical formulation of any one of claims 1 to 20, wherein the antibody that specifically binds to endothelial lipase comprises a heavy chain comprising an amino acid sequence at least 90% identical to SEQ ID NO:9 and a light chain comprising an amino acid sequence at least 90% identical to SEQ ID NO: 10.

44. The pharmaceutical formulation of any one of claims 1 to 20, wherein the antibody that specifically binds to endothelial lipase comprises a heavy chain comprising SEQ ID NO:9 and a light chain comprising SEQ ID NO:10.

45. The pharmaceutical formulation of any one of claims 1 to 44, further comprising histidine.

46. The pharmaceutical formulation of claim 45, wherein the pharmaceutical formulation comprises about 5 mM to about 40 mM histidine.

47. The pharmaceutical formulation of claim 45, wherein the pharmaceutical formulation comprises about 20 mM histidine.

48. The pharmaceutical formulation of claim 45, wherein the pharmaceutical formulation comprises about 10 mM histidine.

49. The pharmaceutical formulation of any one of claims 45 to 48, wherein the histidine is a hydrochloride salt.

50. The pharmaceutical formulation of any one of claims 1 to 49, further comprising a surfactant.

51. The pharmaceutical formulation of claim 50, wherein the surfactant is polysorbate 20, polysorbate 60, polysorbate 80.

52. The pharmaceutical formulation of claim 50, wherein the surfactant is polysorbate 80.

53. The pharmaceutical formulation of claim 52, wherein the pharmaceutical formulation comprises from about 0.005% to about 0.05% polysorbate 80.

54. The pharmaceutical formulation of claim 52, wherein the pharmaceutical formulation comprises from about 0.02% to about 0.04% polysorbate 80.

55. The pharmaceutical formulation of claim 52, wherein the pharmaceutical formulation comprises about 0.02% polysorbate 80.

56. The pharmaceutical formulation of claim 52, wherein the pharmaceutical formulation comprises about 0.04% polysorbate 80.

57. The pharmaceutical formulation of any one of claims 1 to 56, wherein the pharmaceutical formulation has a pH less than 7.0.

58. The pharmaceutical formulation of any one of claims 1 to 56, wherein the pharmaceutical formulation has a pH of about 5.0 to about 7.0.

59. The pharmaceutical formulation of any one of claims 1 to 58, wherein the pharmaceutical formulation has a pH of about 6.0.

60. The pharmaceutical formulation of any one of claims 1 to 59, wherein the formulation has a viscosity of ≤30 cP at 18° C.

61. The pharmaceutical formulation of any one of claims 1 to 59, wherein the formulation has a viscosity of ≤20 cP at 18° C.

62. The pharmaceutical formulation of any one of claims 1 to 59, wherein the formulation has a viscosity of ≤15 cP at 18° C.

63. The pharmaceutical formulation of any one of claims 1 to 62, wherein the formulation has an osmolality of about 260 to about 500 mOsm/kg.

64. The pharmaceutical formulation of any one of claims 1 to 62, wherein the formulation has an osmolality of about 440 to about 460 mOsm/kg.

65. The pharmaceutical formulation of any one of claims 1 to 64, wherein the pharmaceutical formulation is suitable for injection.

66. The pharmaceutical formulation of claim 65, wherein the pharmaceutical formulation is suitable for intravenous injection.

67. The pharmaceutical formulation of claim 65, wherein the pharmaceutical formulation is suitable for subcutaneous injection.

68. The pharmaceutical formulation of claim 65, wherein the pharmaceutical formulation is suitable for intramuscular injection.

69. The pharmaceutical formulation of any one of claims 1 to 68, wherein the pharmaceutical formulation is reconstituted from a lyophilized formulation.

70. The pharmaceutical formulation of any of one claims 1 to 69, wherein the pharmaceutical formulation is a liquid formulation.

71. The pharmaceutical formulation of any one of claims 1 to 70, wherein the pharmaceutical formulation is suitable for administration with an injection device.

72. The pharmaceutical formulation of claim 71, wherein the injection device is an autoinjector device.

73. The pharmaceutical formulation of claim 71 or 72, wherein the injection device is a large volume bolus injector.

74. The pharmaceutical formulation of claim 71 or 72, wherein the injection device is a pre-filled syringe.

75. A method for treating cardiovascular disease in a mammalian subject in need thereof comprising administering to the subject the pharmaceutical formulation of any one of claims 1 to 74.

76. A method for preventing cardiovascular disease in a mammalian subject in need thereof comprising administering to the subject the pharmaceutical formulation of any one of claims 1 to 74.

77. A method for reducing atherosclerosis in a mammalian subject in need thereof comprising administering to the subject the pharmaceutical formulation of any one of claims 1 to 74.

78. A method for reducing the risk of cardiovascular death, myocardial infarction, stroke, and/or coronary revascularization in a mammalian subject with prior acute coronary syndrome comprising administering to the subject the pharmaceutical formulation of any one of claims 1 to 74.

79. A method for preventing a secondary cardiovascular event in a mammalian subject in need thereof comprising administering to the subject the pharmaceutical formulation of any one of claims 1 to 74.

80. A method for reducing the risk of a major adverse cardiovascular event in a mammalian subject in need thereof comprising administering to the subject the pharmaceutical formulation of any one of claims 1 to 74.

81. A method for increasing the concentration of high-density lipoprotein in the blood of a mammalian subject in need thereof comprising administering to the subject the pharmaceutical formulation of any one of claims 1 to 74.

82. A method for increasing one or more clinical endpoints related to high-density lipoprotein in a mammalian subject in need thereof comprising administering to the subject the pharmaceutical formulation of any one of claims 1 to 74; wherein the clinical endpoints are HDL-c blood concentration, HDL particle number, HDL particle size, HDL phospholipid blood concentration, ApoA1 blood concentration and cholesterol efflux capacity.

83. A method for decreasing inflammation in a mammalian subject in need thereof comprising administering to the subject the pharmaceutical composition of any one of claims 1 to 74.

84. A method for preventing inflammation in a mammalian subject in need thereof comprising administering to the subject the pharmaceutical composition of any one of claims 1 to 74.

85. The method of any one of claims 75 to 84, wherein the pharmaceutical formulation is administered with an injection device.

86. The method of claim 85, wherein the injection device is an autoinjector device.

87. The method of claim 85 or 86, wherein the injection device is a large volume bolus injector.

88. The method of claim 85 or 86, wherein the injection device is a pre-filled syringe.

89. The method of any one of claims 75 to 88, wherein the mammalian subject is a human.

90. A lyophilized pharmaceutical formulation comprising i) an antibody that specifically binds to endothelial lipase and ii) arginine hydrochloride; wherein the lyophilized pharmaceutical formulation can be reconstituted to a concentration of about 100 mg/mL to about 160 mg/mL of an antibody that specifically binds to endothelial lipase, and about 120 mM to about 260 mM arginine salt; and wherein the lyophilized pharmaceutical formulation is pharmaceutically acceptable.

91. A lyophilized pharmaceutical formulation comprising i) an antibody that specifically binds to endothelial lipase, ii) arginine hydrochloride, iii) histidine or a salt thereof, and iv) polysorbate 80; wherein the lyophilized pharmaceutical formulation can be reconstituted to a concentration of about 125 mg/mL antibody that specifically binds to endothelial lipase, about 160 mM arginine hydrochloride, about 20 mM histidine or a salt thereof and about 0.04% polysorbate 80; and wherein the lyophilized pharmaceutical formulation is pharmaceutically acceptable.

92. A lyophilized pharmaceutical formulation comprising i) an antibody that specifically binds to endothelial lipase, ii) arginine hydrochloride, iii) histidine or a salt thereof, and iv) polysorbate 80; wherein the lyophilized pharmaceutical formulation can be reconstituted to a concentration of about 125 mg/mL antibody that specifically binds to endothelial lipase, about 220 mM arginine hydrochloride, about 20 mM histidine or a salt thereof and about 0.04% polysorbate 80; and wherein the lyophilized pharmaceutical formulation is pharmaceutically acceptable.

93. A lyophilized pharmaceutical formulation comprising i) an antibody that specifically binds to endothelial lipase, ii) arginine hydrochloride, iii) histidine or a salt thereof, and iv) polysorbate 80; wherein the lyophilized pharmaceutical formulation can be reconstituted to a concentration of about 100 mg/mL antibody that specifically binds to endothelial lipase, about 160 mM arginine hydrochloride, about 20 mM histidine or a salt thereof and about 0.04% polysorbate 80; and wherein the lyophilized pharmaceutical formulation is pharmaceutically acceptable.

94. The lyophilized pharmaceutical formulation of any one of claims 90-93, wherein the pharmaceutical formulation can be reconstituted to a pH of about 6.0.

95. A liquid pharmaceutical formulation comprising about 125 mg/mL of an antibody that specifically binds to endothelial lipase, about 220 mM arginine hydrochloride, about 10 mM histidine or a salt thereof and about 0.02% polysorbate 80, and is pharmaceutically acceptable.

96. A liquid pharmaceutical formulation comprising about 100 mg/mL of an antibody that specifically binds to endothelial lipase, about 220 mM arginine hydrochloride, about 10 mM histidine or a salt thereof and about 0.02% polysorbate 80, and is pharmaceutically acceptable.

97. A liquid pharmaceutical formulation comprising about 125 mg/mL of an antibody that specifically binds to endothelial lipase, about 220 mM arginine hydrochloride, about 20 mM histidine or a salt thereof and about 0.02% polysorbate 80, and is pharmaceutically acceptable.

98. A liquid pharmaceutical formulation comprising about 100 mg/mL of an antibody that specifically binds to endothelial lipase, about 220 mM arginine hydrochloride, about 20 mM histidine or a salt thereof and about 0.02% polysorbate 80, and is pharmaceutically acceptable.

99. A liquid pharmaceutical formulation comprising about 50 mg/mL of an antibody that specifically binds to endothelial lipase, about 80 mM arginine hydrochloride, about 10 mM histidine or a salt thereof and about 0.02% polysorbate 80, and is pharmaceutically acceptable.

100. The liquid pharmaceutical formulation of any one of claims 95-99, wherein the pharmaceutical formulation has a pH of about 6.0.
